(19) United States
(12) Patent Application Publication
(10) Pub. No.: US 2026/0130771 A1
(43) Pub. Date: May 14, 2026

(54) ORTHOPEDIC SPINAL SURGICAL IMPLANT AND METHOD OF USE

(71) Applicant: FutureSpine Inc., Naples, FL (US)

(72) Inventor: Mark FRENKEL, Naples, FL (US)

(21) Appl. No.: 18/670,649

(22) Filed: May 21, 2024

Related U.S. Application Data
(60) Provisional application No. 63/460,330, filed on Apr. 19, 2023.
(60) Provisional application No. 63/528,912, filed on Jul. 25, 2023.

(51) Int. Cl. A61F 2/44; A61B 17/02; A61F 2/46

(52) U.S. Cl. CPC A61F 2/446; A61F 2/4611; A61B 2017/0256

(57) ABSTRACT

An intervertebral device for orthopedic spinal surgery which allows bone cement to be intercalated with the device as the cement is introduced through the device and into the vertebral body. This may be achieved by a number of mechanisms, including an embedded deployable cannula or external instrumentation. The intervertebral device includes a body having top, bottom and side surfaces. A working port is provided through the sidewall. A cannula is operated via the port and passes through an aperture formed in at least one of the top/bottom surfaces. The cannula can be deployed through an angled guide; through a vertical guide, operated by an actuator passing through a guide originating through the sidewall; by a plate as a subassembly; and as subassembly comprising a series of hollow pins carried by a plate. Cannula deployment can be accomplished filling an interior void or a mechanical device.

### CROSS REFERENCE TO RELATED APPLICATIONS

This Non-Provisional Application claims the benefit of:
A) U.S. Provisional Patent Application Ser. No. 63/460,330, filed on Apr. 19, 2023, and
B) U.S. Provisional Patent Application Ser. No. 63/528,912, filed on Jul. 25, 2023,
both of which are incorporated herein in their entireties by reference.

### FIELD OF THE INVENTION

The present disclosure generally relates to medical tooling to aid in an Orthopedic Spinal Surgical Procedure of a patient. The medical tooling includes an implantable interbody device (alternatively referred to as a “graft”, “cage”, or “spacer”) which includes specific elements that can be used to inject polymethylmethacrylate (“PMMA”) or other surgical “bone cement” or “cement” into a vertebral body. The device allows a more permanent fixation of two vertebral bodies together as well as provides strength augmentation of the bone.

### BACKGROUND OF THE INVENTION

In spinal fusion surgery there are several different anatomic approaches to the spine. Some of these include posterior, lateral, or anterior approaches where the surgeon accesses the disc space directly. In interbody fusion procedures, a portion of the disc space is cleared out and an interbody device is inserted in its place. In the lumbar spine specifically, it is commonplace for the surgeon to also place pedicle screws from a posterior approach to increase stability of the construct. In addition, patients with osteopenia or osteoporosis are an increased risk of vertebral body fracture and graft subsidence and so vertebroplasty is occasionally performed in conjunction with the procedure. This can be done through fenestrated pedicle screws which act as a cannula through which to pass bone cement, or by placing a trocar directly into the vertebral body.

Graft subsidence is a sinking of a body with a higher elasticity modulus, such as a graft, a cage, a spacer, and the like, in a body characterized by a lower elasticity modulus, such as a vertebral body, resulting in three-dimensional changes of the spinal geometry. A magnitude of subsidence is directly proportional to a load pressure and to a difference between the elasticity modules, but inversely proportional to the area of the graft-bed interface. Both biological and mechanical qualities of the graft-bed interface are important for the subsidence process. Any excessive subsidence decreases the interbody space and produces both local and general kyphotization of the spine. This may cause destabilization of the screw-plate and/or screw-bone interfaces, such as pulling-out, altered angulation, or breakage of the screws, or any other undesirable destabilizing change.

What is desired is an interbody implant which can be used to place bone cement into the vertebral body without necessitating a separate approach and also allows the bone cement to intercalate or attach to the implant, thus reducing graft subsidence.

### BRIEF DESCRIPTION OF THE INVENTION

The present disclosure generally relates to medical device for use in an Orthopedic Spinal Surgical Procedure of a patient. The medical device includes an implantable interbody device for the spine. The interbody device contains either deployable cannula which can penetrate through the adjacent vertebral bodies and be used to introduce bone cement into the adjacent vertebral bodies, or channels for the placement of cannula into the bodies.

In a general scope of the present invention, a medical device arrangement includes:
An implantable vertebral interbody device comprising at least one passageway for placement of bone cement into adjacent vertebral bodies.

In a second aspect, at least one deployable cannula is employed to pierce one or both of the vertebral body endplates of the implantable vertebral interbody device.

In another aspect, the implantable vertebral interbody device includes at least one channel, wherein the at least one channel is angled towards adjacent vertebral bodies.

In yet another aspect, the implantable vertebral interbody device includes at least one channel, wherein the at least one channel is used as a conduit for placing bone cement into the adjacent vertebral bodies.

In yet another aspect, the bone cement is polymethylmethacrylate.

In yet another aspect, bone graft can be placed instead of or in addition to the bone cement

In yet another aspect, the implantable vertebral interbody device includes at least one channel, wherein the at least one channel is used as a conduit for instrumentation used for placing the bone cement into the adjacent vertebral bodies.

In yet another aspect, the device enables intercalation of the bone cement with the implantable vertebral interbody device.

In yet another aspect, the medical device comprising:
An implantable vertebral interbody device comprising a body having a deployable interbody locking member guide in communication with an interbody lock actuating control conduit,
wherein an interbody locking member is slideable located within the deployable interbody locking member guide,
wherein, in operation, an interbody locking member actuator is provided through the interbody lock actuating control conduit, when actuated, the interbody locking member actuator causes the interbody locking member to rise beyond a respective vertebral contacting surface.

In yet another aspect, a body of the implantable vertebral interbody device comprising an upper vertebral contacting surface, a lower vertebral contacting surface, and a sidewall extending between the upper vertebral contacting surface and the lower vertebral contacting surface.

In yet another aspect, the upper vertebral contacting surface and the lower vertebral contacting surface are planar in shape.

In yet another aspect, the upper vertebral contacting surface and the lower vertebral contacting surface are convex in shape.

In yet another aspect, the interbody lock actuating control conduit is provided as a bore.

In yet another aspect, the interbody lock actuating control conduit is provided as a bore extending inward from the sidewall.

In yet another aspect, the interbody lock actuating control conduit is provided as a bore, wherein the bore terminates at the deployable interbody locking member guide.

In yet another aspect, the interbody lock actuating control conduit is provided as a bore, wherein the bore intersects with the deployable interbody locking member guide.

In yet another aspect, the interbody lock actuating control conduit is provided as a bore, wherein the bore intersects with and extends beyond the deployable interbody locking member guide.

In yet another aspect, the interbody lock actuating control conduit is provided as a bore extending inward from the sidewall towards a central location of the body.

In yet another aspect, the interbody lock actuating control conduit includes an access orifice.

In yet another aspect, the interbody lock actuating control conduit includes an access orifice located in the sidewall of the implantable vertebral interbody device.

In yet another aspect, the interbody locking member is a solid component.

In yet another aspect, the interbody locking member is a tubular component.

In yet another aspect, the interbody locking member is a cannula.

In yet another aspect, the interbody locking member includes an actuating end.

In yet another aspect, the interbody locking member includes an actuating end, wherein the actuating end is pointed.

In yet another aspect, the interbody locking member includes an actuating end, wherein the actuating end is conically shaped.

In yet another aspect, the interbody locking member includes an actuating end, wherein the actuating end is tapered.

In yet another aspect, the interbody locking member includes a translating end.

In yet another aspect, the interbody locking member includes a translating end, wherein the translating end is tapered.

In yet another aspect, the interbody locking member actuator is a mechanical component.

In yet another aspect, the interbody locking member actuator is a gas.

In yet another aspect, the interbody locking member actuator includes a tapered actuating end.

In yet another aspect, the interbody locking member includes a tapered actuating end.

In yet another aspect, the tapered actuating end of the interbody locking member actuator engages with the tapered actuating end of the interbody locking member causing the interbody locking member to be repositioned beyond the vertebral contacting surface.

In yet another aspect, the tapered actuating end of the interbody locking member actuator engages with the tapered actuating end of the interbody locking member causing the interbody locking member to be slideably repositioned beyond the vertebral contacting surface.

In yet another aspect, the interbody locking member is snugly slideably assembled within the interbody lock actuating control conduit.

In yet another aspect, a composition in a fluid state is provided between the interbody locking member and the interbody lock actuating control conduit.

In yet another aspect, a composition in a fluid state is provided between the interbody locking member and the interbody lock actuating control conduit providing a general gaseous seal.

In yet another aspect, in use, the implantable vertebral interbody device is inserted between two adjacent vertebras.

In yet another aspect, in use, following insertion of the implantable vertebral interbody device between two adjacent vertebras, the interbody locking member is actuated.

In yet another aspect, in use, following insertion of the implantable vertebral interbody device between two adjacent vertebras, the interbody locking member is deployed, positioning an actuating end to contact the respective vertebra.

In yet another aspect, in use, following insertion of the implantable vertebral interbody device between two adjacent vertebras, the interbody locking member is deployed, causing an actuating end of the interbody locking member to pierce the respective vertebra.

In yet another aspect, in use, following insertion of the implantable vertebral interbody device between two adjacent vertebras, the interbody locking member is deployed by applying an actuating force to the translating end, causing the actuating end of the interbody locking member to pierce the respective vertebra.

In yet another aspect, in use, following insertion of the implantable vertebral interbody device between two adjacent vertebras, the interbody locking member is deployed by applying an actuating force to the translating end by applying a force to the actuating end of the interbody lock actuating member, causing the actuating end of the interbody locking member to pierce the respective vertebra.

In yet another aspect, in use, following insertion of the implantable vertebral interbody device between two adjacent vertebras, the interbody locking member is deployed by applying the actuating force to the translating end by providing a gas pressure through the interbody lock actuating control conduit, causing the actuating end of the interbody locking member to pierce the respective vertebra.

In yet another aspect, in use, following insertion of the implantable vertebral interbody device between two adjacent vertebra and deployment of the interbody locking member, a stabilization material is dispensed into the vertebral body.

In yet another aspect, in use, following insertion of the implantable vertebral interbody device between two adjacent vertebra and deployment of the interbody locking member, a stabilization material is dispensed into the vertebral body, wherein the stabilization material is dispensed through the interbody lock actuating control conduit and the deployable interbody locking member guide.

In yet another aspect, in use, following insertion of the implantable vertebral interbody device between two adjacent vertebra and deployment of the interbody locking member, a stabilization material is dispensed into the vertebral body, wherein the stabilization material is dispensed through the interbody lock actuating control conduit and the deployable interbody locking member guide.

In yet another aspect, in use, following insertion of the implantable vertebral interbody device between two adjacent vertebra and deployment of the interbody locking member, a stabilization material is dispensed into the vertebral body, wherein the stabilization material is dispensed through the interbody lock actuating control conduit and a conduit or passageway formed through the deployable interbody locking member.

In yet another aspect, in use, following insertion of the implantable vertebral interbody device between two adjacent vertebra and deployment of the interbody locking member, a stabilization material is dispensed into the vertebral body, wherein the stabilization material is bone cement.

In yet another aspect, in use, following insertion of the implantable vertebral interbody device between two adjacent vertebra and deployment of the interbody locking member, a stabilization material provides a backing within the vertebral body to avoid subsidence of the graft.

In yet another aspect, in use, following insertion of the implantable vertebral interbody device between two adjacent vertebra and deployment of the interbody locking member, a stabilization material is dispensed into the vertebral body, wherein the stabilization material is a volume of bone graft material.

In a variant of the present invention, the medical device comprising:
An implantable vertebral interbody device comprising a body having at least one an interbody lock actuating control conduit extending from sidewall of the body through a vertebral contacting surface of the body.

In yet another aspect, an interbody locking cannula is inserted through the interbody lock actuating control conduit.

In yet another aspect, an interbody locking cannula is slideably inserted through the interbody lock actuating control conduit.

In yet another aspect, the interbody locking cannula is a solid member.

In yet another aspect, the interbody locking cannula is a solid member and used to retain the implantable vertebral interbody device in position.

In yet another aspect, the interbody locking cannula is a solid member and only used to retain the implantable vertebral interbody device in position.

In yet another aspect, the interbody locking cannula is a tubular member.

In yet another aspect, an engaging end of the interbody locking cannula is pointed.

In yet another aspect, an actuating end of the interbody locking cannula is planar.

In yet another aspect, an actuating end of the interbody locking cannula is concave.

In yet another aspect, in operation, following insertion of the implantable vertebral interbody device between two adjacent vertebras, the interbody locking cannula is inserted through the interbody lock actuating control conduit until an engaging end engages with the adjacent vertebra.

In yet another aspect, in operation, following insertion of the implantable vertebral interbody device between two adjacent vertebra, the interbody locking cannula is inserted through the interbody lock actuating control conduit until an engaging end engages with the adjacent vertebra, once engaged, a stabilization material can be provided through a conduit of the interbody locking cannula to be dispensed into the vertebral body.

In yet another aspect, in operation, following insertion of the implantable vertebral interbody device between two adjacent vertebra, the interbody locking cannula is inserted through the interbody lock actuating control conduit until an engaging end engages with the adjacent vertebra, once engaged, a stabilization material can be provided through a conduit of the interbody locking cannula to be dispensed into the vertebral body, wherein the stabilization material is a volume of bone cement.

In yet another aspect, in operation, following insertion of the implantable vertebral interbody device between two adjacent vertebra, the interbody locking cannula is inserted through the interbody lock actuating control conduit until an engaging end engages with the adjacent vertebra, once engaged, a volume of a stabilization material can be provided through a conduit of the interbody locking cannula to be dispensed into the vertebral body.

In yet another aspect, the stabilization material is bone cement.

In yet another aspect, volume of a stabilization material creates a mass of stabilization material connecting the interbody device to the vertebral body.

In yet another aspect, in operation, following insertion of the implantable vertebral interbody device between two adjacent vertebra, the interbody locking cannula is inserted through the interbody lock actuating control conduit until an engaging end engages with the adjacent vertebra, once engaged, a stabilization material can be provided through a conduit of the interbody locking cannula to be dispensed into the vertebral body, wherein the stabilization material is a volume of bone graft material.

In another variant of the present invention, the medical device comprising:
An implantable vertebral interbody device comprising a body having an upper vertebral contacting surface, a lower vertebral contacting surface, and a sidewall extending between the upper vertebral contacting surface and the lower vertebral contacting surface;
at least one aperture through one of the upper vertebral contacting surface and the lower vertebral contacting surface;
a pin subassembly comprising at least one pin carried by a pin supporting base, each at least one pin being in registration with a respective aperture of the at least one aperture;
a port through the sidewall,
wherein outward positioning of the pin subassembly is accomplished by one of: (a) a mechanical device inserted through the port, (b) a mechanical device or operated through the port, or (c) an inflatable device operated through the port.

In yet another aspect, wherein each of the upper vertebral contacting surface and the lower vertebral contacting surface is planar.

In yet another aspect, wherein each of the upper vertebral contacting surface and the lower vertebral contacting surface is substantially planar.

In yet another aspect, wherein each of the upper vertebral contacting surface and the lower vertebral contacting surface is curved.

In yet another aspect, wherein each of the upper vertebral contacting surface and the lower vertebral contacting surface has a dome shape.

In yet another aspect, wherein outward positioning of the pin subassembly is accomplished by the mechanical device inserted through the port.

In yet another aspect, wherein outward positioning of the pin subassembly is accomplished by a mechanism included in the interbody device operated through the port.

In yet another aspect, wherein outward positioning of the pin subassembly is accomplished by operating/filling the inflatable device through the port.

In yet another aspect, wherein outward positioning of the pin subassembly is accomplished by the inflatable device operated through the port, wherein the inflatable device is hydraulically operated.

In yet another aspect, wherein outward positioning of the pin subassembly is accomplished by operating/filling the inflatable device operated through the port, wherein the inflatable device is pneumatically operated.

In yet another aspect, wherein outward positioning of the pin subassembly is accomplished by operating/filling the inflatable device through the port, wherein the inflatable device is removable.

In yet another aspect, the implantable vertebral interbody device further comprising at least one aperture through the upper vertebral contacting surface and at least one aperture through the lower vertebral contacting surface;
wherein the pin subassembly comprising at least one pin carried by the pin supporting base is an upper pin subassembly, each at least one pin being in registration with a respective aperture of the at least one aperture extending through the upper vertebral contacting surface;
a second pin subassembly comprising at least one pin carried by a lower pin supporting base, each at least one lower pin being in registration with a respective aperture of the at least one aperture extending through the lower vertebral contacting surface;
wherein outward positioning of each of the upper pin subassembly and the lower pin subassembly is accomplished by one of the mechanical device inserted through the port or the inflatable device operated through the port.

In yet another aspect, the upper pin subassembly further comprising a plurality of pins carried by the pin supporting base, each of the pins is in registration with a respective aperture extending through the upper vertebral contacting surface.

In yet another aspect, the lower pin subassembly further comprising a plurality of pins carried by the pin supporting base, each of the pins is in registration with a respective aperture extending through the lower vertebral contacting surface.

In yet another aspect, one or more pins of the at least one pin further comprising an orifice passing therethrough.

In yet another aspect, the pins further comprise an orifice passing therethrough.

In yet another aspect, the pins are cannulae, the cannulae further comprise an orifice passing therethrough and a channel located at a base end thereof, wherein the channel enables passage of material from an exterior surface of the cannulae and into the orifice defined by an interior surface of the cannulae.

In yet another aspect, the upper pin supporting base and the lower pin supporting base include a fluid delivery channel.

In yet another aspect, the upper pin supporting base and the lower pin supporting base include the fluid delivery channel, wherein the fluid delivery channel is in registration with at least a portion of a series of cannula deployment member cannula supporting bores.

In yet another aspect, the upper pin supporting base and the lower pin supporting base include the fluid delivery channel, wherein the fluid delivery channel is in registration with each of the series of cannula deployment member cannula supporting bores.

In yet another aspect, the upper pin supporting base and the lower pin supporting base include the fluid delivery channel, wherein the fluid delivery channel is fabricated by forming a groove in a body of the upper pin supporting base and the lower pin supporting base and inserting a delivery conduit fill member into the groove.

In yet another aspect, the upper pin supporting base and the lower pin supporting base include the fluid delivery channel, wherein the fluid delivery channel is fabricated by forming a groove into a sidewall of the body of the upper pin supporting base and the lower pin supporting base and inserting the delivery conduit fill member into the groove.

In yet another aspect, each of the series of cannula deployment member cannula supporting bores is formed prior to insertion of the delivery conduit fill member into the groove.

In yet another aspect, each of the series of cannula deployment member cannula supporting bores is formed following insertion of the delivery conduit fill member into the groove.

In yet another aspect, a pointed end of a plurality of the at least one pin defines an arched surface.

In yet another aspect, a pointed end of a plurality of the at least one pin defines an arched surface, wherein the arched shape follows a contour of a common shape of a contacting surface of a vertebral body.

In yet another aspect, an outer surface of the pin supporting base is planar.

In yet another aspect, the outer surface of the pin supporting base is substantially planar.

In yet another aspect, the outer surface of the pin supporting base is generally planar.

In yet another aspect, the outer surface of the pin supporting base has an arched shape.

In yet another aspect, the outer surface of the pin supporting base has a domed shape.

In yet another aspect, an inner surface of the pin supporting base is planar.

In yet another aspect, the inner surface of the pin supporting base is substantially planar.

In yet another aspect, the inner surface of the pin supporting base is generally planar.

In yet another aspect, the inner surface of the pin supporting base has an arched shape.

In yet another aspect, the inner surface of the pin supporting base has a domed shape.

In yet another aspect, a mechanical control subassembly is located between the upper pin supporting base and the lower pin supporting base.

In yet another aspect, the mechanical control subassembly employs a threaded member to expand and contract a distance between the upper pin supporting base and the lower pin supporting base.

In yet another aspect, the mechanical control subassembly employs a first cannula deploying wedge member threadably engaged with the threaded member, wherein exterior surface of the first cannula deploying wedge member contacts interior surfaces of the upper pin supporting base and the lower pin supporting base causing expansion and contraction of the distance between the upper pin supporting base and the lower pin supporting base.

In yet another aspect, the mechanical control subassembly employs a first cannula deploying wedge member and a second cannula deploying wedge member threadably engaged with the threaded member, wherein an exterior surface of the first cannula deploying wedge member and an exterior surface of the second cannula deploying wedge member contact interior surfaces of the upper pin supporting base and the lower pin supporting base causing expansion and contraction of the distance between the upper pin supporting base and the lower pin supporting base.

In yet another aspect, wherein the first cannula deploying wedge member threadably engages with a first oriented threaded portion of the threaded member and the second cannula deploying wedge member threadably engages with a second, opposite oriented threaded portion of the threaded member, wherein the exterior surface of the first cannula deploying wedge member and the exterior surface of the second cannula deploying wedge member contact interior surfaces of the upper pin supporting base and the lower pin supporting base causing expansion and contraction of the distance between the upper pin supporting base and the lower pin supporting base.

In yet another aspect, wherein the first cannula deploying wedge member threadably engages with a first oriented threaded portion of the threaded member and the second cannula deploying wedge member threadably engages with a second, opposite oriented threaded portion of the threaded member, wherein the exterior surface of the first cannula deploying wedge member and the exterior surface of the second cannula deploying wedge member contact concave shaped interior surfaces of the upper pin supporting base and the lower pin supporting base causing expansion (during an expansion process, the first and second cannula deploying wedge members separate from one another) and contraction (during a contraction process, the first and second cannula deploying wedge members are drawn towards one another) of the distance between the upper pin supporting base and the lower pin supporting base.

In yet another aspect, wherein the first cannula deploying wedge member threadably engages with a first oriented threaded portion of the threaded member and the second cannula deploying wedge member threadably engages with a second, opposite oriented threaded portion of the threaded member, wherein the exterior surface of the first cannula deploying wedge member and the exterior surface of the second cannula deploying wedge member contact convex shaped interior surfaces of the upper pin supporting base and the lower pin supporting base causing expansion (during an expansion process, the first and second cannula deploying wedge members are drawn towards one another) and contraction (during a contraction process, the first and second cannula deploying wedge members separate from one another) of the distance between the upper pin supporting base and the lower pin supporting base.

In yet another aspect, the threaded member can be operated by a mechanical tool.

In yet another aspect, the threaded member can be operated by a removable mechanical tool.

In yet another aspect, the threaded member can be operated by a non-circular shaped tip of the mechanical tool.

In yet another aspect, the threaded member can be operated by a hexagonally shaped tip of the mechanical tool.

In yet another aspect, the threaded member can be operated by a tip of the mechanical tool having a 6-point star-shaped pattern.

In yet another aspect, the threaded member can be operated by a Phillips head tip of the mechanical tool.

In yet another aspect, the pins of the series of pins are all of the same height.

In yet another aspect, the pins of the series of pins have varying heights.

In yet another aspect, the pins of the series are arranged having different orientations of the points of thereof.

In yet another aspect, the upper pin supporting base and the lower pin supporting base remain in position upon completion of the implanting of the intervertebral device.

In yet another aspect, the upper pin supporting base and the lower pin supporting base are removed prior to completion of the implanting of the intervertebral device.

In yet another aspect, wherein the upper pin subassembly is an upper central pin subassembly;
wherein the second pin subassembly is a lower central pin subassembly;
the implantable vertebral interbody device further comprising:
   an upper peripheral pin subassembly comprising at least one pin carried by the peripheral pin supporting base, each at least one pin being in registration with a respective peripheral aperture of the at least one aperture extending through the upper vertebral contacting surface, wherein the upper peripheral pin subassembly is generally radially outward respective to the upper central pin subassembly; and
   a lower peripheral pin subassembly comprising at least one pin carried by the peripheral pin supporting base, each at least one pin being in registration with a respective peripheral aperture of the at least one aperture extending through the lower vertebral contacting surface, wherein the lower peripheral pin subassembly is generally radially outward respective to the lower central pin subassembly;
wherein outward positioning of each of the upper central pin subassembly and the lower central pin subassembly is accomplished by one of a first mechanical device inserted through the port or a first inflatable device operated through a first port,
wherein outward positioning of each of the upper peripheral pin subassembly and the lower peripheral pin subassembly is accomplished by one of a second mechanical device inserted through the port or a second inflatable device operated through a second port.

In yet another aspect, the first port and the second port are the same port.

In yet another aspect, the first port and the second port are distinct from one another.

In yet another aspect, wherein outward positioning of the upper and lower central pin subassemblies are accomplished by a first mechanical device operated through the first port.

In yet another aspect, wherein outward positioning of the upper and lower peripheral pin subassemblies is accomplished by a second, outer mechanical device operated through the second port.

In yet another aspect, wherein outward positioning of the upper and lower central pin subassemblies is accomplished by the first mechanical device operated through the first port.

In yet another aspect, wherein outward positioning of the upper and lower peripheral pin subassemblies is accomplished by the second mechanical device operated through the second port.

In yet another aspect, the first mechanical device can be a member that is rotated.

In yet another aspect, the second mechanical device can be a member that is rotated.

In yet another aspect, the first mechanical device can be a rotating cam.

In yet another aspect, the second mechanical device can be a rotating cam.

In yet another aspect, the first mechanical device can be an expanding and contracting mechanism.

In yet another aspect, the second mechanical device can be an expanding and contracting mechanism.

In yet another aspect, the first mechanical device and the second mechanical device are integrated into a single instrument.

In yet another aspect, wherein outward positioning of the upper and lower central pin subassemblies are accomplished by a first inflatable device operated through the first port, wherein the first inflatable device is inflated hydraulically.

In yet another aspect, wherein outward positioning of the upper and lower peripheral pin subassemblies is accomplished by a second, outer inflatable device operated through the second port, wherein the second, outer inflatable device is inflated hydraulically.

In yet another aspect, wherein outward positioning of the upper and lower central pin subassemblies is accomplished by the first inflatable device operated through the first port, wherein the first inflatable device is a removable hydraulically inflated member.

In yet another aspect, wherein outward positioning of the upper and lower peripheral pin subassemblies is accomplished by the second inflatable device operated through the second port, wherein the second inflatable device is a removable hydraulically inflated member.

In yet another aspect, wherein outward positioning of the upper and lower central pin subassemblies are accomplished by a first inflatable device operated through the first port, wherein the first inflatable device is pneumatically inflated.

In yet another aspect, wherein outward positioning of the upper and lower peripheral pin subassemblies is accomplished by a second, outer inflatable device operated through the second port, wherein the second, outer inflatable device is pneumatically inflated.

In yet another aspect, wherein outward positioning of the upper and lower central pin subassemblies is accomplished by the first inflatable device operated through the first port, wherein the first inflatable device is a removable pneumatically inflated member.

In yet another aspect, wherein outward positioning of the upper and lower peripheral pin subassemblies is accomplished by the second inflatable device operated through the second port, wherein the second inflatable device is a removable pneumatically inflated member.

In yet another aspect, a volume of bone cement is delivered through conduits formed through at least one pin of at least one of the pin subassemblies.

In yet another aspect, a volume of bone cement is delivered through conduits formed through at least one pin of at least one of the peripheral pin subassemblies.

In yet another aspect, a volume of bone cement is delivered through conduits formed through at least one pin of at least one of each of the upper peripheral pin subassembly and the lower peripheral pin subassembly.

In yet another aspect, the volume of bone cement is delivered through a respective port in the sidewall of the interbody device.

In yet another aspect, a volume of bone graft material is delivered through conduits formed through at least one pin of at least one of the pin subassemblies.

In yet another aspect, a volume of bone graft material is delivered through conduits formed through at least one pin of at least one of the central pin subassemblies.

In yet another aspect, a volume of bone graft material is delivered through conduits formed through at least one pin of at least one of each of the upper central pin subassembly and the lower central pin subassembly.

In yet another aspect, the volume of bone graft material is delivered through a respective port in the sidewall of the interbody device.

In yet another aspect, an outer peripheral edge of the central pin supporting base slideably engages with an inner peripheral edge of the peripheral pin supporting base.

In yet another aspect, a thickness along at least a portion of the outer peripheral edge of the central pin is greater than a thickness along at least a mating portion of the inner peripheral edge of the peripheral pin supporting base.

In yet another aspect, a thickness along the outer peripheral edge of the central pin is greater than a thickness along a mating portion of the inner peripheral edge of the peripheral pin supporting base.

In yet another aspect, a thickness along the outer peripheral edge of the peripheral pin is greater than a thickness along a mating portion of the inner peripheral edge of the central pin supporting base.

In accordance with the present invention, a method comprising steps of:
inserting an implantable vertebral interbody device between adjacent vertebras, the implantable vertebral interbody device comprising:
a body having an upper vertebral contacting surface, a lower vertebral contacting surface, and a sidewall extending between the upper vertebral contacting surface and the lower vertebral contacting surface,
a port through the sidewall,
at least one orifice passing through at least one of the upper vertebral contacting surface and the lower vertebral contacting surface; and
a moveable cannula passing through a respective orifice of the at least one orifice passing through the at least one of the upper vertebral contacting surface and the lower vertebral contacting surface;
deploying the cannula by applying a force to a first, force receiving end of the cannula, wherein the force inserts a second, engaging end of the cannula into a facing surface of the respective vertebra; and
dispensing a stabilization material through the cannula.

In a second aspect, the step of dispensing a stabilization material through the cannula dispenses the stabilization material into the respective vertebra.

In yet another aspect, the method further comprising a step of dispensing the stabilization material into the respective vertebra of the adjacent vertebras.

In another aspect, the step of dispensing a stabilization material through the cannula dispenses the stabilization material into the respective vertebra, wherein the stabilization material stabilizes the respective vertebra in an area proximate the respective surface of the implantable vertebral interbody device.

In yet another aspect, the step of dispensing the stabilization material through the cannula is accomplished by using bone cement as the stabilization material.

In yet another aspect, the method further comprising a step of withdrawing the cannula from the vertebra.

In yet another aspect, the method further comprising a step of passing the cannula through the port through the sidewall.

In yet another aspect, the step of deploying the cannula by applying a force to a first, force receiving end of the cannula is accomplished by utilizing a mechanically applied force.

In yet another aspect, the step of deploying the cannula by applying a force to a first, force receiving end of the cannula is accomplished by utilizing a pneumatically applied force.

In yet another aspect, the second, engaging end of the cannula further comprising a pointed shape.

In yet another aspect, the second, engaging end of the cannula further comprising a pointed shape, method further comprising a step of using the pointed shape of the cannula to penetrate the respective vertebra of the adjacent vertebras.

In yet another aspect, the implantable vertebral interbody device comprising further comprising a cannula deploying member, the cannula deploying member being engaged with the cannula.

In yet another aspect, the implantable vertebral interbody device comprising further comprising a cannula deploying member, the cannula deploying member being engaged with the cannula, the method further comprising a step of using the cannula deploying member to apply the force to a first, force receiving end of the cannula.

In yet another aspect, the implantable vertebral interbody device comprising further comprising a cannula deploying member, the cannula deploying member being engaged with the cannula, the method further comprising a step of using the cannula deploying member to apply the force to a first, force receiving end of the cannula.

In yet another aspect, the implantable vertebral interbody device further comprising at least one cannula arranged to pass through one of the upper vertebral contacting surface and the lower vertebral contacting surface, the method further comprising a step of:
applying an outwardly directed vertical force to each force receiving end of each at least one upper cannula to penetrate the respective vertebra of the adjacent vertebras.

In yet another aspect, the implantable vertebral interbody device further comprising at least one upper cannula arranged to pass through the upper vertebral contacting surface and at least one lower cannula arranged to pass through the lower vertebral contacting surface, the method further comprising steps of:
applying an upward force to each force receiving end of each at least one upper cannula to penetrate the respective vertebra of the adjacent vertebras; and
applying a downward force to each force receiving end of each at least one lower cannula to penetrate the respective vertebra of the adjacent vertebras.

In yet another aspect, the implantable vertebral interbody device further comprising a cannula subassembly comprising at least one cannula carried by a cannula supporting base, each at least one cannula being in registration with a respective aperture of the at least one aperture, the method further comprising a step of:
outwardly positioning the pin subassembly by one of a mechanical device inserted through the port or a pressure generating device operated through the port.

In yet another aspect, the implantable vertebral interbody device is as described in the method of use described herein.

In yet another aspect, the implantable vertebral interbody device is as described in the method of use described herein, wherein the cannula additionally passes through the port through the sidewall.

In yet another aspect, the implantable vertebral interbody device is as described in the method of use described herein, the second, engaging end of the cannula further comprising a pointed shape.

In yet another aspect, the implantable vertebral interbody device is as described in the method of use described herein, the upper vertebral contacting surface and the lower vertebral contacting surface having a planar horizontal surface.

In yet another aspect, the implantable vertebral interbody device is as described in the method of use described herein, the upper vertebral contacting surface and the lower vertebral contacting surface having an outwardly convex surface.

In yet another aspect, the implantable vertebral interbody device is as described in the method of use described herein, at least one element of the implantable vertebral interbody device further comprising a conduit providing transfer of the stabilization material to the cannula.

In accordance with the present invention, a method of locating and retaining an interbody implant between two vertebra, the method comprising steps of:
Inserting an implantable vertebral interbody device between two adjacent vertebra, the implantable vertebral interbody device comprising an intervertebral device body having an upper vertebral contacting surface, a lower vertebral contacting surface, and a sidewall extending between the upper vertebral contacting surface and the lower vertebral contacting surface,
deploying at least one of bone cement and a bone graft material through a passageway provided, wherein the passageway initiates through the sidewall and terminates through at least one of the upper vertebral contacting surface and the lower vertebral contacting surface.

In yet another aspect, the method comprising a step of dispensing bone cement.

In yet another aspect, the method comprising a step of dispensing bone graft material.

In yet another aspect, the method further comprising a step of deploying at least one cannula to provide mechanical retention of the implantable vertebral interbody device between the two adjacent vertebras.

In yet another aspect, the cannula comprising a tubular element having a pointed insertion end.

In yet another aspect, the method further comprising a step of deploying at least one cannula to provide mechanical retention of the implantable vertebral interbody device between the two adjacent vertebras, wherein the cannula is slideably inserted through the passageway.

In yet another aspect, the method further comprising a step of deploying at least one cannula to provide mechanical retention of the implantable vertebral interbody device between the two adjacent vertebra, and following the deployment of the at least one cannula, the method further comprising a step of dispensing at least one of bone cement and bone graft material.

In yet another aspect, the method further comprising a step of deploying at least one cannula to deter destabilization of a screw-plate and/or screw-bone interfaces, and following the deployment of the at least one cannula, the method further comprising a step of dispensing at least one of bone cement and bone graft material.

In yet another aspect, the method further comprising a step of deploying at least one cannula to deter pulling out of one or more screws, prevent altered angulation of the screws, prevent altered angulation of the interbody device, and/or prevent breakage of screws, and following the deployment of the at least one cannula, the method further comprising a step of dispensing at least one of bone cement and bone graft material.

In yet another aspect, the method further comprising a step of deploying at least one cannula to deter destabilization of a screw-plate and/or screw-bone interfaces, and following the deployment of the at least one cannula, the method further comprising a step of dispensing at least one of bone cement and bone graft material into the vertebral body.

In yet another aspect, the method further comprising a step of positioning the cannula via an interbody lock actuating member.

In yet another aspect, the interbody lock actuating member being operated through an interbody lock actuating control conduit, wherein the interbody lock actuating control conduit extends from the sidewall to at least an intersection with a deployable interbody locking member guide.

In yet another aspect, the interbody lock actuating member being slideably operated through the interbody lock actuating control conduit, wherein the interbody lock actuating control conduit extends from the sidewall to at least an intersection with the deployable interbody locking member guide.

In yet another aspect, the deployable interbody lock actuating member being an elongated mechanical component.

In yet another aspect, the deployable interbody lock actuating member being a compressed gas.

In yet another aspect, the deployable interbody lock actuating member being a non-compressible fluid.

In yet another aspect, the deployable interbody locking member guide extends between a central location within the intervertebral device body and one of the upper vertebral contacting surface and the lower vertebral contacting surface.

In accordance with the present invention, a method of locating and retaining an interbody implant between two vertebra, the method comprising steps of:
Inserting an implantable vertebral interbody device between two adjacent vertebras, the implantable vertebral interbody device comprising:
   an intervertebral device body having an upper vertebral contacting surface, a lower vertebral contacting surface, and a sidewall extending between the upper vertebral contacting surface and the lower vertebral contacting surface,
   a pin subassembly comprising at least one pin carried by a pin supporting base, each at least one pin being in registration with a respective aperture of the at least one aperture;
deploying the pin subassembly to a position locating the at least one pin through the respective aperture and external of the respective aperture of the at least one aperture provided through the respective one of the upper vertebral contacting surface and the lower vertebral contacting surface.

In yet another aspect, the intervertebral device body further comprising at least one port formed through the sidewall.

In yet another aspect, the method further comprising a step of deploying the pin subassembly by inserting an actuator through the port formed through the sidewall.

In yet another aspect, the actuator is a mechanical device, wherein the method further comprising a step of rotating the actuator to deploy the pin subassembly.

In yet another aspect, the actuator is a mechanical device, wherein the method further comprising a step of rotating the actuator about an elongated axis of a shaft of the actuator to deploy the pin subassembly.

In yet another aspect, the actuator is a mechanical device, wherein the method further comprising a step of pivoting the actuator to deploy the pin subassembly.

In yet another aspect, the actuator is a mechanical device, wherein the method further comprising a step of pivoting the actuator about a point along the elongated axis of the shaft of the actuator to deploy the pin subassembly, wherein the actuator acts as a lever.

In yet another aspect, the actuator is a mechanical device, wherein the method further comprising a step of pivoting the actuator about an axis that is substantially perpendicular to the elongated axis of the shaft of the actuator to deploy the pin subassembly.

In yet another aspect, the actuator is a mechanical device, wherein the method further comprising a step of pivoting the actuator about an axis that is substantially perpendicular to the elongated axis of the shaft of the actuator to deploy the pin subassembly, wherein the actuator acts as a lever.

In yet another aspect, the actuator is a hydraulic device, wherein the method further comprising a step of filling the actuator with a fluid to deploy the pin subassembly.

In yet another aspect, the actuator is a hydraulic device, wherein the hydraulic device is removable.

In yet another aspect, the actuator is a pneumatic device, wherein the method further comprising a step of filling the actuator with a gas to deploy the pin subassembly.

In yet another aspect, the actuator is a pneumatic device, wherein the pneumatic device is removable.

In yet another aspect, the pin subassembly is an upper pin subassembly comprising at least one pin carried by an upper pin supporting base, wherein each at least one pin being in registration with a respective aperture of the at least one aperture passing through the upper vertebral contacting surface, the implantable vertebral interbody device further comprising a lower pin subassembly comprising at least one pin carried by a lower pin supporting base, each at least one pin being in registration with a respective aperture of the at least one aperture passing through the lower vertebral contacting surface, the method further comprising a step of:
deploying the upper pin subassembly to a position locating the at least one pin through the respective aperture and external of the respective aperture of the at least one aperture provided through the upper vertebral contacting surface; and
deploying the lower pin subassembly to a position locating the at least one pin through the respective aperture and external of the respective aperture of the at least one aperture provided through the lower vertebral contacting surface.

In yet another aspect, the actuator comprising a central pneumatic device and a peripheral pneumatic device.

In yet another aspect, the upper pin subassembly is an upper central pin subassembly, the implantable vertebral interbody device further comprising an upper peripheral pin subassembly comprising at least one pin carried by an upper peripheral pin supporting base, each at least one pin being in registration with a respective peripheral aperture of the at least one aperture passing through the upper vertebral contacting surface, the method further comprising a step of:
deploying at least one of the upper central pin subassembly and the upper peripheral pin subassembly to a position locating the at least one pin through the respective aperture and external of the respective aperture of the at least one aperture provided through the upper vertebral contacting surface.

In yet another aspect, the lower pin subassembly is a lower central pin subassembly, the implantable vertebral interbody device further comprising a lower peripheral pin subassembly comprising at least one pin carried by a lower peripheral pin supporting base, each at least one pin being in registration with a respective peripheral aperture of the at least one aperture passing through the lower vertebral contacting surface, the method further comprising a step of:
deploying at least one of the lower central pin subassembly and the lower peripheral pin subassembly to a position locating the at least one pin through the respective aperture and external of the respective aperture of the at least one aperture provided through the lower vertebral contacting surface.

In yet another aspect, the actuator comprising the central pneumatic device and the peripheral pneumatic device, wherein the central pneumatic device is arranged to deploy at least one of the central pin subassemblies and the peripheral pneumatic device is arranged to deploy at least one of the peripheral pin subassemblies.

In yet another aspect, the actuator comprising the central pneumatic device and the peripheral pneumatic device, wherein the central pneumatic device is arranged to deploy at least one of the central pin subassemblies, wherein the peripheral pneumatic device is arranged to deploy at least one of the peripheral pin subassemblies, the method further comprising a step of independently actuating the central pneumatic device and the peripheral pneumatic device respective to one another.

In yet another aspect, the actuator comprising the central pneumatic device and the peripheral pneumatic device, wherein the central pneumatic device is arranged to deploy at least one of the central pin subassemblies, wherein the peripheral pneumatic device is arranged to deploy at least one of the peripheral pin subassemblies, the method further comprising a step of simultaneously actuating the central pneumatic device and the peripheral pneumatic device with one another.

In yet another aspect, the actuator comprising the central pneumatic device and the peripheral pneumatic device, wherein the central pneumatic device is arranged to deploy the upper central pin subassembly and the lower central pin subassembly and the peripheral pneumatic device is arranged to deploy the upper peripheral pin subassembly and the lower peripheral pin subassembly.

In yet another aspect, the actuator is a pneumatic device, wherein the method further comprising a step of filling the peripheral pneumatic device is arranged to deploy at least one of the peripheral pin subassemblies.

In yet another aspect, the actuator is a pneumatic device, wherein the method further comprising a step of filling the peripheral pneumatic device is arranged to deploy the upper peripheral pin subassembly and the lower peripheral pin subassembly.

In yet another aspect, the actuator is a pneumatic device, wherein the method further comprising a step of filling the central pneumatic device is arranged to deploy at least one of the central pin subassemblies.

In yet another aspect, the actuator is a pneumatic device, wherein the method further comprising a step of filling the central pneumatic device is arranged to deploy the upper central pin subassembly and the lower central pin subassembly.

In yet another aspect, the method further comprising a step of removing the pneumatic device following deployment of the respective at least one pin subassembly.

In yet another aspect, one or more of the at least one pin further comprising a passageway.

In yet another aspect, one or more of the at least one pin further comprising a passageway, the method further comprising a step of passing at least one of bone cement and bone graft material through the passageway passing through the respective pin.

In yet another aspect, one or more of the at least one pin further comprising a passageway, the method further comprising a step of passing bone cement through the passageway passing through the respective pin.

In yet another aspect, the method further comprising a step of passing bone cement through the passageway passing through the respective pin/cannula and impregnating the respective bone.

In yet another aspect, the method further comprising a step of passing bone cement through the passageway passing through the respective pin/cannula and impregnating the respective bone, where a portion of the bone cement remains within the respective bone and the another portion remains within the cannula.

In yet another aspect, the method further comprising a step of passing bone cement through the passageway passing through the respective pin/cannula and impregnating the respective bone, where a portion of the bone cement remains within the respective bone and the another portion remains within the vertebral cannula passage extending through the respective intervertebral device body.

In yet another aspect, the method further comprising a step of passing bone cement through the passageway passing through the respective pin/cannula and impregnating the respective bone, where a portion of the bone cement remains within the respective bone and the another portion remains within the vertebral cannula passage extending through the respective intervertebral device body when the cannula is removed.

In yet another aspect, one or more of the at least one pin further comprising a passageway, the method further comprising a step of passing bone graft material through the passageway passing through the respective pin.

In yet another aspect, the actuator is a mechanical device, wherein the method further comprising a step of using the actuator to deploy the upper central pin subassembly and the lower central pin subassembly.

In yet another aspect, the actuator is a mechanical device, wherein the method further comprising a step of moving the actuator to deploy the upper central pin subassembly and the lower central pin subassembly.

In yet another aspect, the actuator is a mechanical device, wherein the method further comprising a step of rotating the actuator to deploy the upper central pin subassembly and the lower central pin subassembly.

In yet another aspect, the actuator is a mechanical device, wherein the method further comprising a step of pivoting the actuator to deploy the upper central pin subassembly and the lower central pin subassembly.

In yet another aspect, the actuator is a mechanical device, wherein the method further comprising a step of expanding the actuator to deploy the upper central pin subassembly and the lower central pin subassembly.

In yet another aspect, the actuator is a mechanical device, wherein the method further comprising a step of using the actuator to deploy the upper peripheral pin subassembly and the lower peripheral pin subassembly.

In yet another aspect, the actuator is a mechanical device, wherein the method further comprising a step of moving the actuator to deploy the upper peripheral pin subassembly and the lower peripheral pin subassembly.

In yet another aspect, the actuator is a mechanical device, wherein the method further comprising a step of rotating the actuator to deploy the upper peripheral pin subassembly and the lower peripheral pin subassembly.

In yet another aspect, the actuator is a mechanical device, wherein the method further comprising a step of pivoting the actuator to deploy the upper peripheral pin subassembly and the lower peripheral pin subassembly.

In yet another aspect, the actuator is a mechanical device, wherein the method further comprising a step of expanding the actuator to deploy the upper peripheral pin subassembly and the lower peripheral pin subassembly.

These and other aspects, features, and advantages of the present invention will become more readily apparent from the attached drawings and the detailed description of the preferred embodiments, which follow.

### BRIEF DESCRIPTION OF THE DRAWINGS

Variants of the invention will hereinafter be described in conjunction with the appended drawings provided to illustrate and not to limit the invention, where like designations denote like elements, and in which:

FIG. 1 presents a perspective view of a first exemplary intervertebral spacer with a deployable cannula mechanism;

FIG. 2 presents a sectioned side elevation view of the first exemplary intervertebral spacer originally introduced in FIG. 1, the section taken along section line 2-2 of FIG. 1, the deployable cannula mechanism illustrated in a retracted configuration;

FIG. 3 presents a sectioned side elevation view of the first exemplary intervertebral spacer originally introduced in FIG. 1, the section taken along section line 2-2 of FIG. 1, the deployable cannula mechanism illustrated in a deployed configuration;

FIG. 4 presents a sectioned side elevation view of the first exemplary intervertebral spacer originally introduced in FIG. 1, the section taken along section line 2-2 of FIG. 1, the first exemplary intervertebral spacer being shown inserted in a location between adjacent vertebra with the deployable cannula mechanism illustrated in a deployed configuration;

FIG. 5 presents an isometric view of a second exemplary intervertebral spacer with an integrated tract for cannula placement;

FIG. 6 presents a sectioned side elevation view of the second exemplary intervertebral spacer originally introduced in FIG. 5, the section taken along section line 6-6 of FIG. 5, the deployable cannula mechanism illustrated in a pre-installation configuration (solid line) and in a deployed configuration (broken line);

FIG. 7 presents a sectioned side elevation view of the second exemplary intervertebral spacer originally introduced in FIG. 5, the section taken along section line 6-6 of FIG. 5, the second exemplary intervertebral spacer being shown inserted in a location between adjacent vertebra with the deployable cannula mechanism illustrated in a deployed configuration;

FIG. 8 presents an isometric view of a third exemplary intervertebral spacer comprising a series of pin subassemblies;

FIG. 9 presents an isometric exploded assembly view of the third exemplary intervertebral spacer originally introduced in FIG. 8;

FIG. 10 presents a sectioned side elevation view of the third exemplary intervertebral spacer originally introduced in FIG. 8, the section taken along section line 10-10 of FIG. 8, wherein the upper and lower deployable pin subassemblies are illustrated in a pre-installation configuration;

FIG. 11 presents a sectioned side elevation view of the third exemplary intervertebral spacer originally introduced in FIG. 8, the section taken along section line 10-10 of FIG. 8, wherein upper and lower central pin subassemblies are illustrated in a pre-installation configuration and upper and lower peripheral pin subassemblies are illustrated in a deployed configuration;

FIG. 12 presents a sectioned side elevation view of the third exemplary intervertebral spacer originally introduced in FIG. 8, the section taken along section line 10-10 of FIG. 8, wherein the upper and lower central pin subassemblies are illustrated in a deployed configuration and the upper and lower peripheral pin subassemblies are illustrated in a deployed configuration;

FIG. 13 presents a sectioned side elevation view of the third exemplary intervertebral spacer originally introduced in FIG. 8, the section taken along section line 10-10 of FIG. 8, wherein upper and lower central pin subassemblies are illustrated in a deployed configuration and upper and lower peripheral pin subassemblies are illustrated in a pre-installation configuration;

FIG. 14 presents a sectioned side elevation view of the third exemplary intervertebral spacer originally introduced in FIG. 8, the section taken along section line 10-10 of FIG. 8, the third exemplary intervertebral spacer being shown inserted in location between adjacent vertebra with the upper and lower central pin subassemblies being illustrated in a deployed configuration and the upper and lower peripheral pin subassemblies being illustrated in a deployed configuration;

FIG. 15 presents an isometric exploded assembly view of a fourth exemplary intervertebral, the illustration displaying components of the assembly;

FIG. 16 presents a front, side isometric view of an exemplary cannula of the fourth exemplary intervertebral originally introduced in FIG. 15;

FIG. 17 presents a side elevation view of the exemplary cannula originally introduced in FIG. 16;

FIG. 18 presents a front elevation view of the exemplary cannula originally introduced in FIG. 16;

FIG. 19 presents an top exploded assembly view of a pin deployment subassembly of the fourth exemplary intervertebral originally introduced in FIG. 15;

FIG. 20 presents a sectioned front elevation assembly view of a cannula assembled to a cannula deployment member of the fourth exemplary intervertebral originally introduced in FIG. 15, the section taken along section line 20-20 of FIG. 19, the illustration detailing a cement flow path through the cannula;

FIG. 21 presents a sectioned side elevation view of the fourth exemplary intervertebral originally introduced in FIG. 15, the section taken along a longitudinal centerline, the fourth exemplary intervertebral spacer being shown inserted in location between adjacent vertebra with an upper cannula subassembly and a lower cannula subassembly being illustrated in a spacer insertion configuration;

FIG. 22 presents a sectioned side elevation view of the fourth exemplary intervertebral originally introduced in FIG. 15, the section taken along a longitudinal centerline, the fourth exemplary intervertebral spacer being shown inserted in location between adjacent vertebra with an upper cannula subassembly and a lower cannula subassembly being illustrated in a deployed configuration using an inflatable deployment subassembly;

FIG. 23 presents a sectioned side elevation view of the fourth exemplary intervertebral originally introduced in FIG. 15, the section taken along a longitudinal centerline, the fourth exemplary intervertebral spacer being shown inserted in location between adjacent vertebra with an upper cannula subassembly and a lower cannula subassembly being illustrated in a deployed configuration wherein bone cement is being initially dispensed therethrough;

FIG. 24 presents a sectioned side elevation view of the fourth exemplary intervertebral originally introduced in FIG. 15, the section taken along a longitudinal centerline, the fourth exemplary intervertebral spacer being shown inserted in location between adjacent vertebra with an upper cannula subassembly and a lower cannula subassembly being illustrated in a deployed configuration wherein bone cement has been adequately dispensed;

FIG. 25 presents a sectioned side elevation view of the fourth exemplary intervertebral originally introduced in FIG. 15, the section taken along a longitudinal centerline, the illustration presenting a first step (deflating an inflatable cannula deploying subassembly) in optionally removing the cannula deployment subassembly;

FIG. 26 presents a sectioned side elevation view of the fourth exemplary intervertebral originally introduced in FIG. 15, the section taken along a longitudinal centerline, the illustration presenting a second step (removing the deflated inflatable cannula deploying subassembly) in optionally removing the cannula deployment subassembly and additionally indicating the steps of removing the upper cannula deployment subassembly;

FIG. 27 presents a sectioned side elevation view of the fourth exemplary intervertebral originally introduced in FIG. 15, the section taken along a longitudinal centerline, the illustration presenting a third step (disengaging the upper cannula deployment subassembly) in optionally removing the cannula deployment subassembly;

FIG. 28 presents a sectioned side elevation view of the fourth exemplary intervertebral originally introduced in FIG. 15, the section taken along a longitudinal centerline, the illustration presenting a fourth step (removing the upper cannula deployment subassembly) in optionally removing the cannula deployment subassembly and additionally indicating the steps of removing the lower cannula deployment subassembly;

FIG. 29 presents a sectioned side elevation view of a fifth exemplary intervertebral, the section taken along a longitudinal centerline, the fifth exemplary intervertebral spacer being shown inserted in location between adjacent vertebra with an upper cannula subassembly and a lower cannula subassembly being illustrated in a spacer insertion configuration; and

FIG. 30 presents a sectioned side elevation view of the fifth exemplary intervertebral originally introduced in FIG. 29, the section taken along a longitudinal centerline, the fourth exemplary intervertebral spacer being shown inserted in location between adjacent vertebra with an upper cannula subassembly and a lower cannula subassembly being illustrated in a deployed configuration using a threaded wedge deployment subassembly.

Like reference numerals refer to like parts throughout the several views of the drawings.

### DETAILED DESCRIPTION OF THE PRESENT INVENTION

Patients may be diagnosed with an anatomical issue requiring spinal surgery, such as a lateral lumbar interbody fusion. This can include a spinal injury, spinal instability, a herniated disc, an osteophyte, or any other anatomical issue or combination thereof.

The current devices used as interbody spacers for lateral and anterior approaches for the spine, most commonly the lumbar spine, have a number of limitations. In patients with osteopenia or osteoporosis, these devices may subside because of stress placed on weak bone. This can sometimes be avoided by performing a vertebroplasty at the time of the index surgery or after the patient has suffered a fracture and subsided device. In addition, these interbody devices frequently do not provide enough stabilization to serve as the only instrumentation in a fusion procedure. Some devices are available which allow for employment of attachment screws inserted into the vertebral bodies, or some separate plating systems with screws are also available, but these are still not as mechanically sound as placing pedicle screws from a separate posterior surgical approach.

The present invention introduces an interbody device which serves to overcome these limitations by allowing cement augmentation of the vertebral bodies above and/or below the interbody device through a channel in the device which also allows the bone cement to anchor into the device for added stabilization.

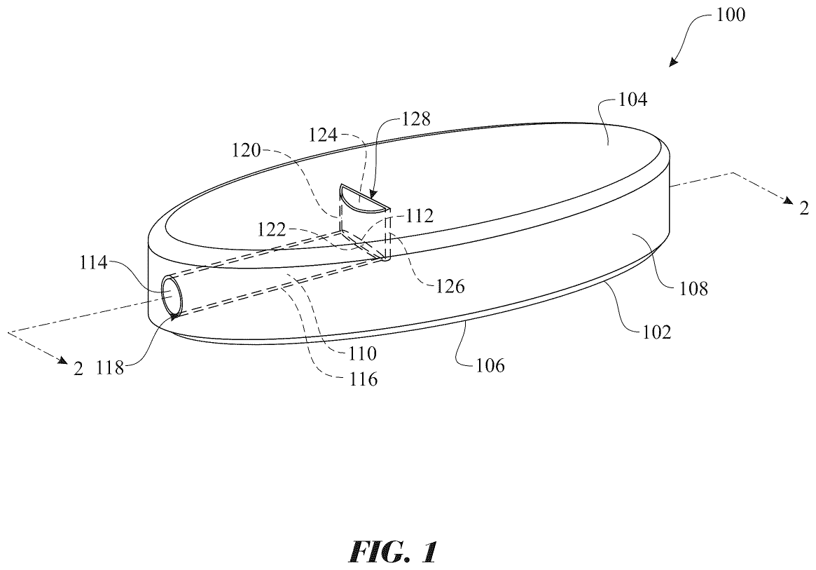

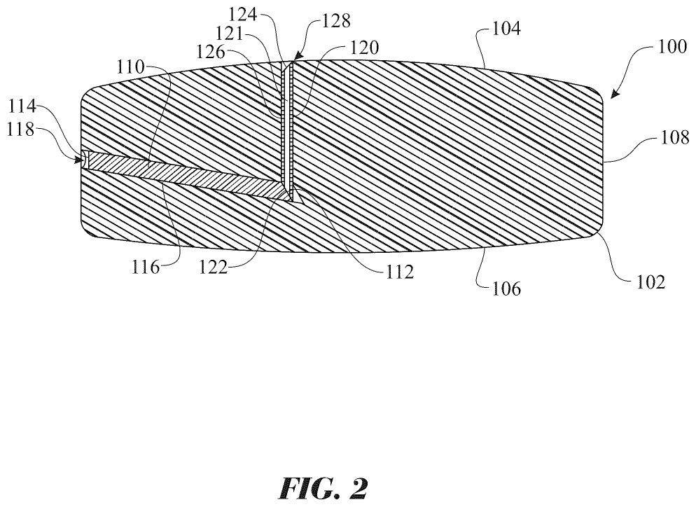

An intervertebral device 100 is introduced in FIG. 1, with cross sectional views illustrating the intervertebral device 100 in use in FIGS. 2 through 4. The intervertebral device 100 includes an intervertebral device body 102, the intervertebral device body 102 having an intervertebral device body sidewall 108 extending between peripheral edges of an intervertebral device body upper vertebral contacting surface 104 and an intervertebral device body lower vertebral contacting surface 106. A deployable interbody locking member 120 is slideably inserted into a deployable interbody locking member guide 126. It is preferred that the deployable interbody locking member 120 is asymmetric to ensure retention of an orientation within the deployable interbody locking member guide 126. The deployable interbody locking member 120 includes a deployable interbody locking member translating end 122 at a first end and a deployable interbody locking member actuating end 124 located at a second, opposite end thereof. The deployable interbody locking member translating end 122 is designed to engage with an interbody lock actuating member 110, wherein when the interbody lock actuating member 110 is actuated, the actuation of the interbody lock actuating member 110 translates to the deployable interbody locking member 120 via the deployable interbody locking member translating end 122. In the exemplary illustration, the interbody lock actuating member 110 includes an interbody lock actuating member translating end 112 and an interbody lock actuating member actuating end 114. The exemplary interbody lock actuating member translating end 112 is angled to act as a wedge against an angled deployable interbody locking member translating end 122 of the deployable interbody locking member 120. The exemplary interbody lock actuating member actuating end 114 is planar or concave to aid in engagement with a deployment tool (not shown). An interbody lock actuating control conduit 116 is formed extending inward from an interbody lock actuating control conduit opening 118 at the intervertebral device body sidewall 108, the interbody lock actuating control conduit 116 extending to intersect with and preferably beyond a deployable interbody locking member guide 126. The interbody lock actuating member 110 is slideably inserted into the interbody lock actuating control conduit 116, wherein the interbody lock actuating member 110 is oriented with the interbody lock actuating member translating end 112 towards and preferably engaging with the deployable interbody locking member translating end 122 of the deployable interbody locking member 120.

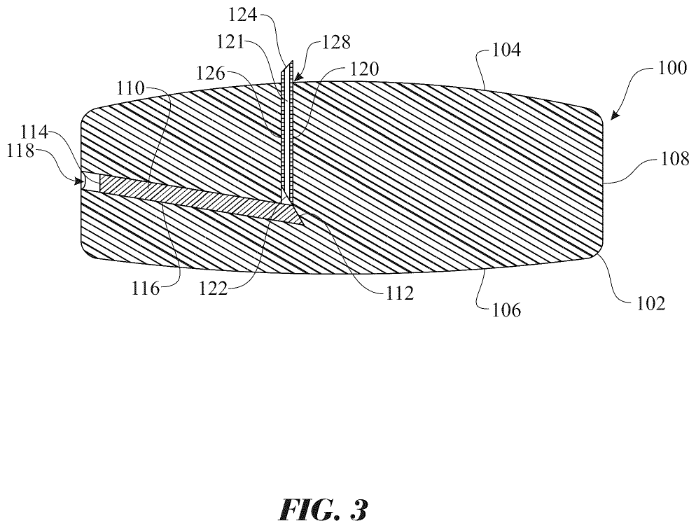

The intervertebral device 100 is prepared for insertion between two adjacent joint members 400, 410 (in the exemplary illustration two adjacent vertebra 402, 412) as illustrated in FIG. 2 by compressing the deployable interbody locking member 120 within the deployable interbody locking member guide 126. An intra-vertebral disc 420 is removed prior to insertion of the intervertebral device 100 between two adjacent joint members 400, 410. The interbody lock actuating member 110 is inserted into the interbody lock actuating control conduit 116, wherein the interbody lock actuating member 110 is oriented with the interbody lock actuating member translating end 112 towards and preferably engaging with the deployable interbody locking member translating end 122 of the deployable interbody locking member 120. The interbody lock actuating member actuating end 114 can be contained within or slightly proud of the interbody lock actuating control conduit opening 118. Following preparation of the insertion location, the intervertebral device 100 is inserted between two adjacent joint members 400, 410. When inserted, the intervertebral device body upper vertebral contacting surface 104 is facing a first vertebrae second joint surface 406 of the first vertebrae 402 and the intervertebral device body lower vertebral contacting surface 106 is facing a second vertebrae first joint surface 414 of the second vertebrae 412. Once the intervertebral device 100 is seated in position between the first vertebrae 402 and the second vertebrae 412, an actuating force is applied to the interbody lock actuating member actuating end 114 of the interbody lock actuating member 110. The interbody lock actuating member translating end 112 of the interbody lock actuating member 110 engages with the deployable interbody locking member translating end 122 of the deployable interbody locking member 120, causing deployment of the deployable interbody locking member 120, as best illustrated in FIG. 3. When the deployable interbody locking member 120 is deployed, the deployable interbody locking member actuating end 124 passes through the deployable interbody locking member guide opening 128 where the deployable interbody locking member actuating end 124 is positioned proud or beyond the intervertebral device body upper vertebral contacting surface 104 and preferably impinging into the adjacent vertebral body 402, 412.

Once the locking member actuating end 124 engages with the first vertebrae second joint surface 406, the interbody lock actuating member 110 can be removed and a volume of stabilization material 139 is dispensed through the interbody lock actuating control conduit 116, continuing through a deployable interbody locking member passageway 121 formed through the deployable interbody locking member 120 through the first vertebrae second joint surface 406 into the vertebral body 402 (as illustrated) or in an alternative arrangement, through the second vertebrae first joint surface 414 and into the vertebral body 412. The stabilization material 139 can be bone cement, such as methyl methacrylate or any other bone cement, bone graft material, any other suitable stabilization material 139, or any combination of suitable stabilization material.

The interbody lock actuating member 110 can be replaced with a pressurized gas and/or fluid. The pressurized gas and/or fluid would be supplied through the interbody lock actuating control conduit opening 118, continuing through the interbody lock actuating control conduit 116 to the deployable interbody locking member translating end 122 of the deployable interbody locking member 120. Pressure from the gas and/or fluid would deploy the deployable interbody locking member 120 through the deployable interbody locking member guide 126.

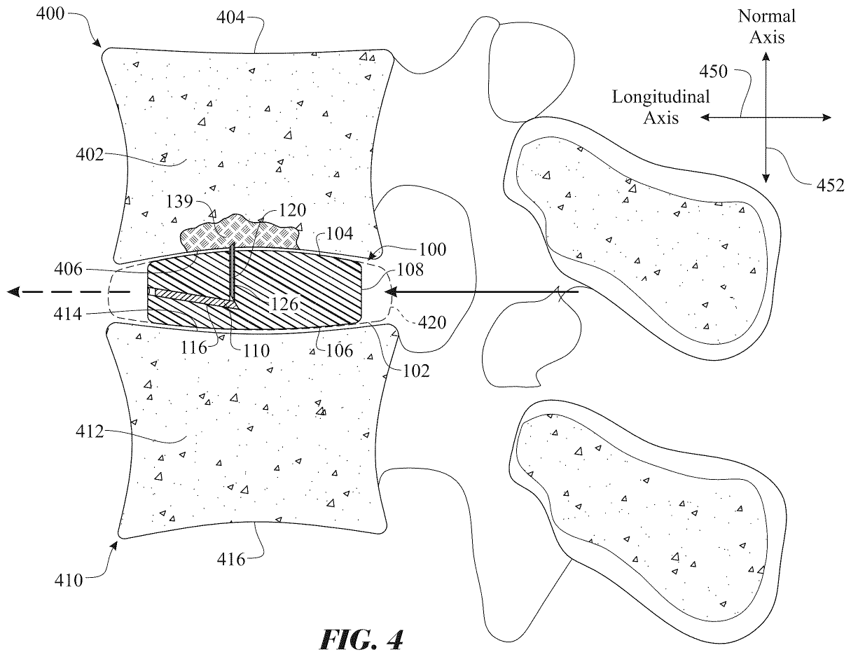

Other features of note in the illustration presented in FIG. 4 include a first vertebrae first joint surface 404 of the first vertebrae 402, a second vertebrae second joint surface 416 of the second vertebrae 412, and orientation references, including a longitudinal axis 450 and a lateral axis 452.

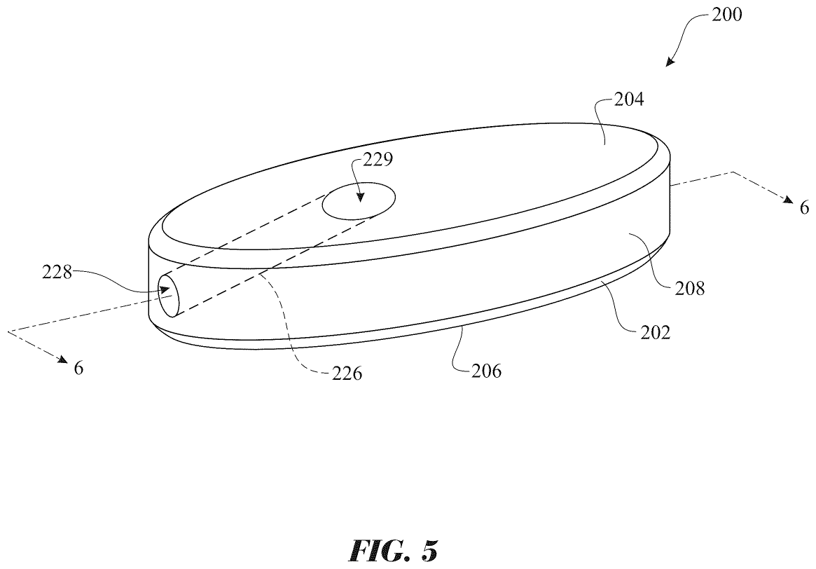

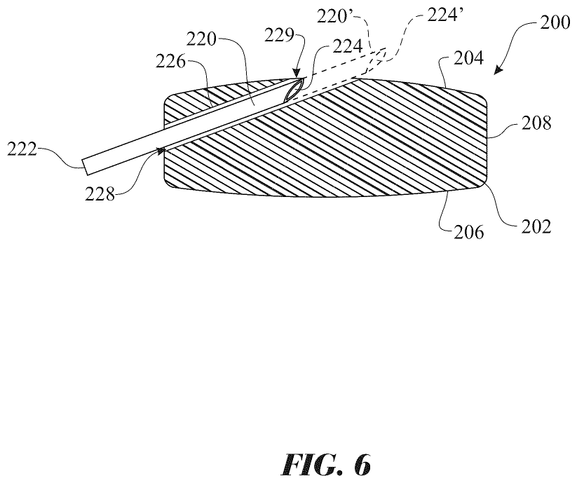

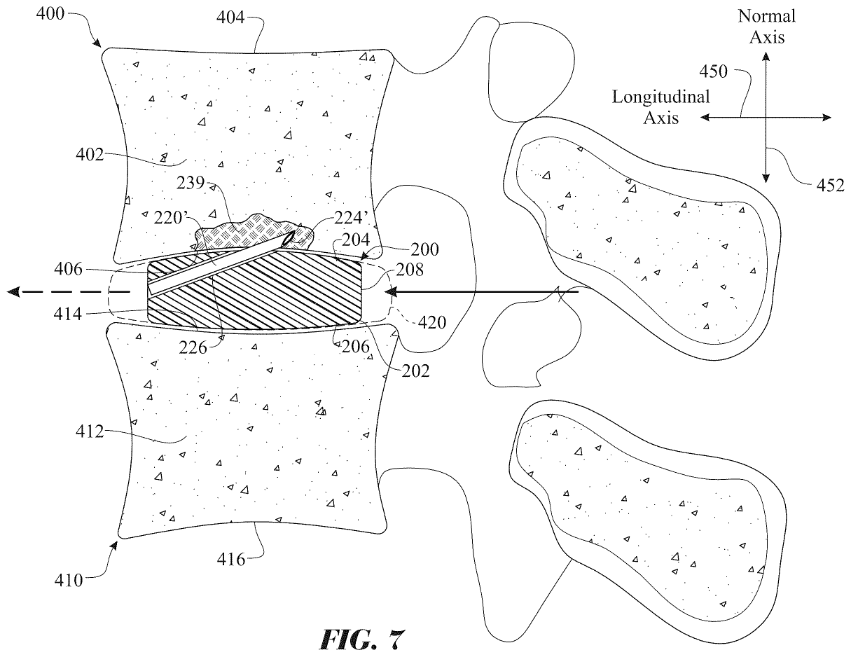

The intervertebral device 100 includes an interbody lock actuating member 110 to operate the deployable interbody locking member 120. An intervertebral device 200, illustrated in FIGS. 5 through 7, eliminates the interbody lock actuating member 110 and the respective interbody lock actuating control conduit 116 from the intervertebral device 100 by orienting an interbody lock actuating control conduit 226 at an angle. Like the intervertebral device 100, the intervertebral device 200 comprising an intervertebral device body 202 including an intervertebral device body sidewall 208 extending between an intervertebral device body upper vertebral contacting surface 204 and an intervertebral device body lower vertebral contacting surface 206. An interbody lock actuating control conduit 226 provides a passageway extending between the intervertebral device body sidewall 208 and one of the intervertebral device body upper vertebral contacting surface 204 (as illustrated) or the intervertebral device body lower vertebral contacting surface 206. An interbody lock actuating control conduit actuating opening 228 is located at a first, actuating end of the interbody lock actuating control conduit 226, passing through the intervertebral device body sidewall 208 and an interbody lock actuating control conduit engaging opening 229 is located at a second, opposite engaging end of the interbody lock actuating control conduit 226, passing through one of the intervertebral device body upper vertebral contacting surface 204 (as illustrated) or the intervertebral device body lower vertebral contacting surface 206. An interbody locking cannula 220 is slideably inserted through the interbody lock actuating control conduit 226. The interbody locking cannula 220 is preferably a tubular member comprising an interbody locking cannula actuating end 222 formed at an actuating end thereof and an interbody locking cannula engaging end 224 formed at an engaging end thereof. The interbody locking cannula actuating end 222 is preferably has a planar or concave shape. The interbody locking cannula engaging end 224 preferably has a pointed shape. A cross sectional view of the intervertebral device 200 is illustrated in FIGS. 6 and 7. Deployment of the interbody locking cannula 220 is best illustrated in FIG. 6, where, shown in solid lines, the interbody locking cannula 220 is placed in an insertion position and, shown in broken lines, the interbody locking cannula (inserted) 220′ is placed in a deployed position extending an interbody locking cannula engaging end (inserted) 224′, piercing the interbody lock actuating control conduit engaging opening 229, breaching the intervertebral device body upper vertebral contacting surface 204 of the intervertebral device body 202, and engaging with the respective facing surface 406, 414 of the respective vertebra 402, 412. The angled relation of the interbody locking cannula (inserted) 220′ respective to a motion of the intervertebral device 200 creates an arrangement of a wedge, retaining the intervertebral device 200 in position.

Once the interbody locking cannula (inserted) 220′ is deployed, a volume of stabilization material 239 can be injected through the interbody locking cannula (inserted) 220′ through the first vertebrae second joint surface 406 and into the first vertebral body 402 (as shown) or through the second vertebrae first joint surface 414 and into the second vertebral body 412 in an alternative. The stabilization material 239 can be bone cement, such as methyl methacrylate or any other bone cement, bone graft material, any other suitable stabilization material 239, or any combination of suitable stabilization material. The interbody locking cannula (inserted) 220′ preferably remains in position.

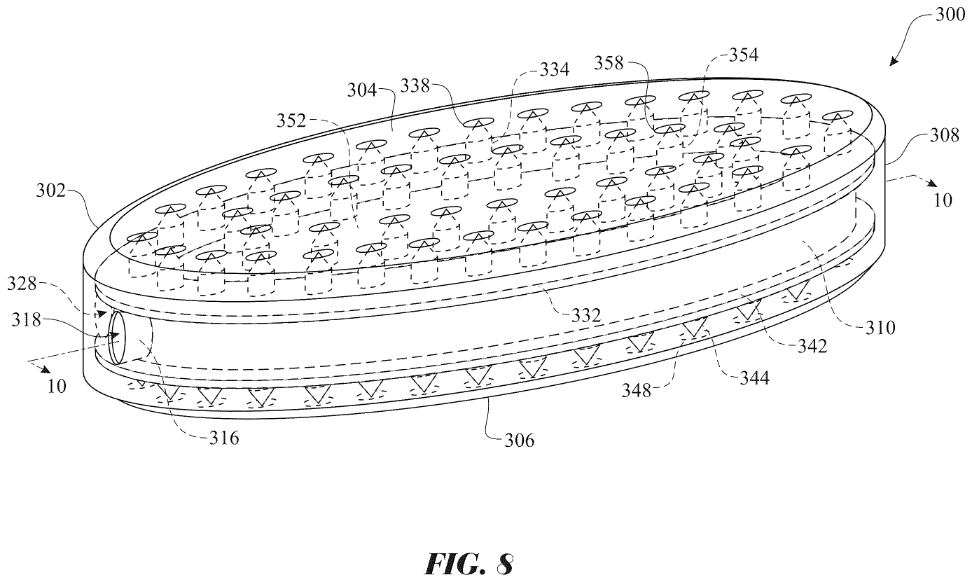

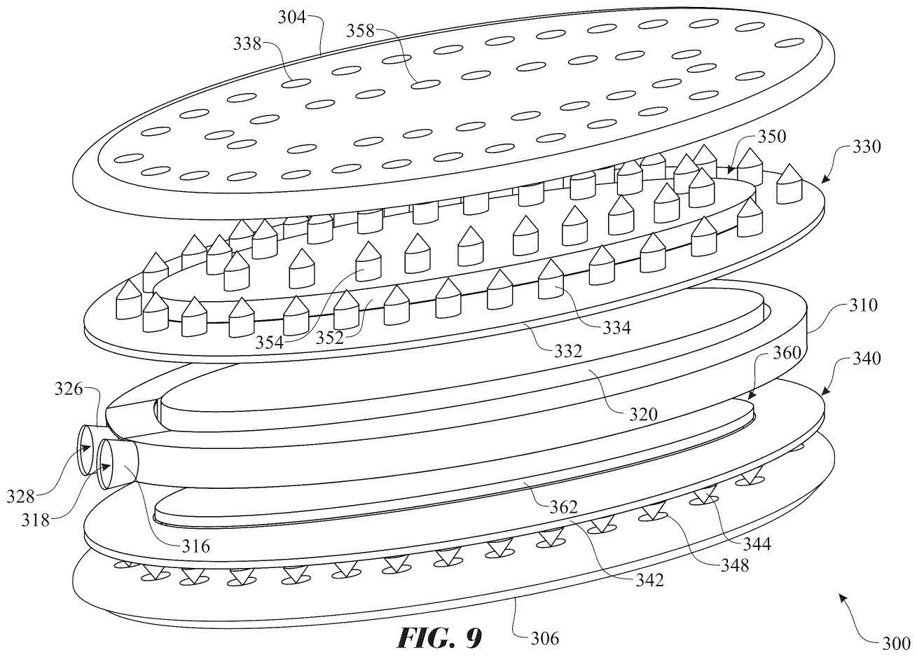

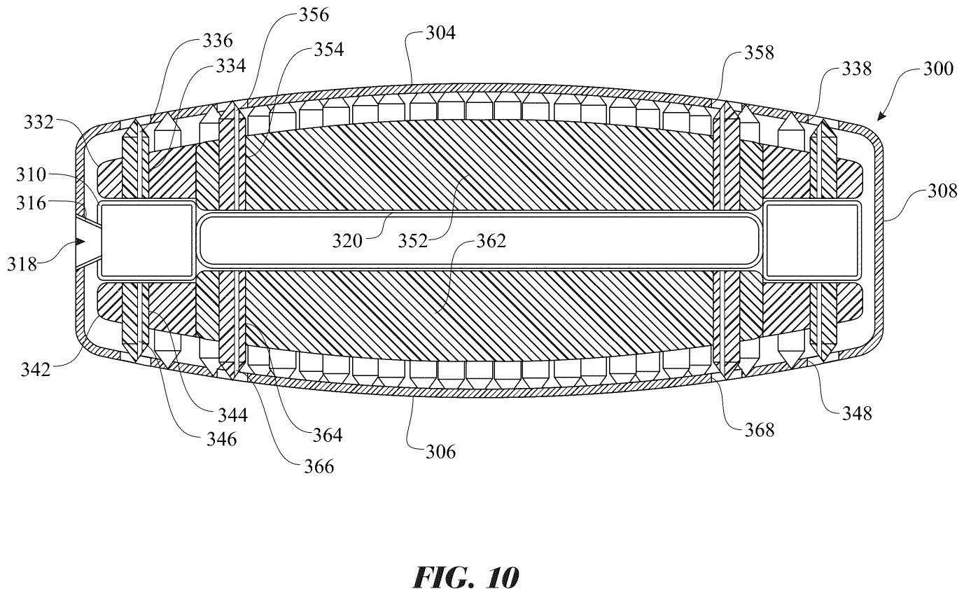

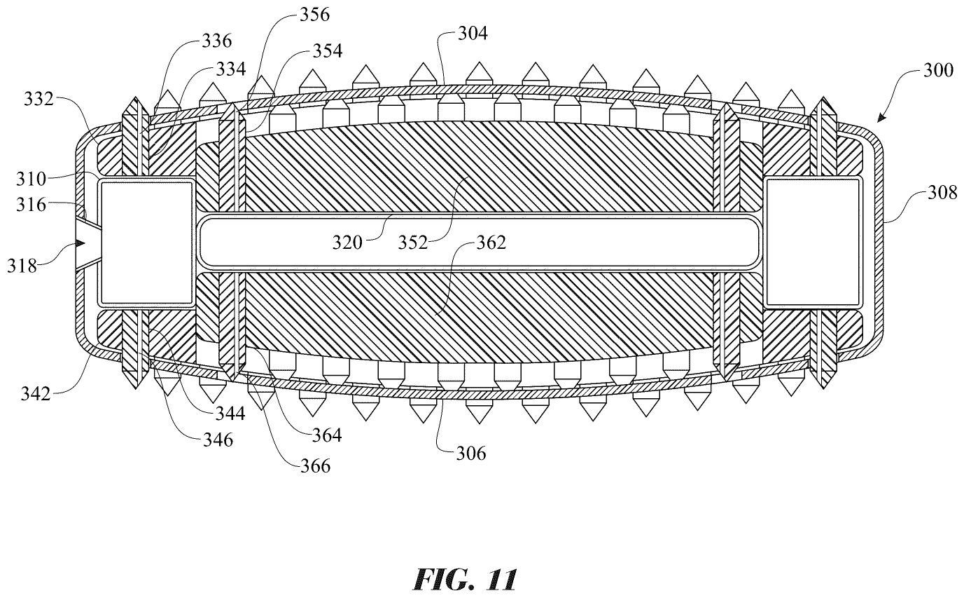

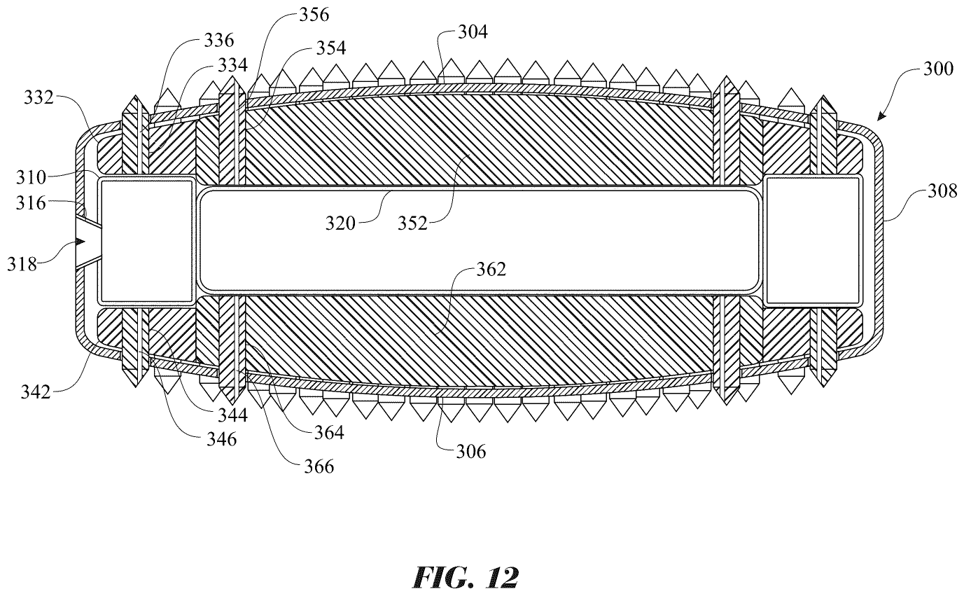

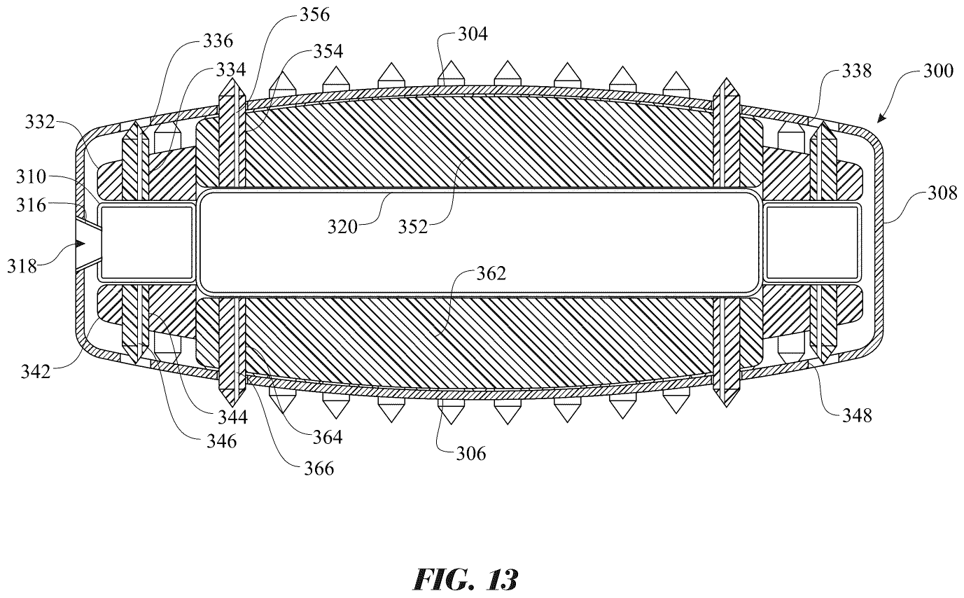

The exemplary intervertebral device 100 and the intervertebral device 200 include an independently operated deployable interbody locking member 120 and interbody locking cannula 220 respectively. An intervertebral device 300, introduced in FIG. 8 and detailed in operation in FIGS. 9 through 14, includes a plurality of retention elements, wherein at least a portion of the retention elements can additionally provide a conduit for dispensing a stabilization material between the intervertebral device 300 and the respective surface 406, 414 of the vertebra 402, 412.

The exemplary intervertebral device 300 includes a plurality of upper peripheral pin passage apertures 338 extending through an intervertebral device body upper vertebral contacting surface 304 of an intervertebral device body 302 of the intervertebral device 300, a plurality of upper central pin passage apertures 358 extending through the intervertebral device body upper vertebral contacting surface 304 of the intervertebral device body 302 of the intervertebral device 300, a plurality of lower peripheral pin passage apertures 348 extending through an intervertebral device body lower vertebral contacting surface 306 of the intervertebral device body 302 of the intervertebral device 300, and a plurality of lower central pin passage apertures 368 extending through an intervertebral device body lower vertebral contacting surface 306 of the intervertebral device body 302 of the intervertebral device 300.

An upper peripheral pin subassembly 330 comprising a plurality of upper peripheral pins 334 carried by an upper peripheral pin supporting base 332 is slideably located within an internal portion of the intervertebral device body 302. Each upper peripheral pin 334 is located in registration with a respective upper peripheral pin passage aperture 338 formed through the intervertebral device body upper vertebral contacting surface 304 of the intervertebral device body 302.

A lower peripheral pin subassembly 340 comprising a plurality of lower peripheral pins 344 carried by a lower peripheral pin supporting base 342 is slideably located within an internal portion of the intervertebral device body 302. Each lower peripheral pin 344 is located in registration with a respective lower peripheral pin passage aperture 348 formed through the intervertebral device body lower vertebral contacting surface 306 of the intervertebral device body 302.

An upper central pin subassembly 350 comprising a plurality of upper central pins 354 carried by an upper central pin support base 352 is slideably located within an internal portion of the intervertebral device body 302. Each upper central pin 354 is located in registration with a respective upper central pin passage aperture 358 formed through the intervertebral device body upper vertebral contacting surface 304 of the intervertebral device body 302.

A lower central pin subassembly 360 comprising a plurality of lower central pins 364 carried by a lower central pin support base 362 is slideably located within an internal portion of the intervertebral device body 302. Each lower central pin 364 is located in registration with a respective lower central pin passage aperture 368 formed through the intervertebral device body lower vertebral contacting surface 306 of the intervertebral device body 302.

The upper peripheral pin subassembly 330 and the upper central pin subassembly 350 are preferably slideably assembled respective to one another. An external peripheral edge of the upper central pin subassembly 350 is positioned against an internal peripheral edge of the upper peripheral pin subassembly 330. A thickness of the upper peripheral pin supporting base 332 and a thickness of the upper central pin support base 352 are such to retain engagement therebetween when the upper peripheral pin supporting base 332 and/or the upper central pin support base 352 are deployed into an engagement position.

The lower peripheral pin subassembly 340 and the lower central pin subassembly 360 are preferably slideably assembled respective to one another. An external peripheral edge of the lower central pin subassembly 360 is positioned against an internal peripheral edge of the lower peripheral pin subassembly 340. A thickness of the lower peripheral pin supporting base 342 and a thickness of the lower central pin support base 362 are such to retain engagement therebetween when the lower peripheral pin supporting base 342 and/or the lower central pin support base 362 are deployed into an engagement position.

In the exemplary embodiment, the upper peripheral pin subassembly 330 and the upper central pin subassembly 350 are mirrored respective to one another and the lower peripheral pin subassembly 340 and the lower central pin subassembly 360 are mirrored respective to one another.

In the exemplary intervertebral device 300, a peripheral pin operating inflatable member 310 is arranged to deploy each of the upper peripheral pin subassembly 330 and the lower peripheral pin subassembly 340. A peripheral pin operating inflatable member fill tube 316 provides a filling medium passageway from the intervertebral device body sidewall 308 via a peripheral pin operating inflatable member fill passageway 318 to an interior of the peripheral pin operating inflatable member 310. As a filling medium is transferred to the peripheral pin operating inflatable member 310, the peripheral pin operating inflatable member 310 expands, deploying the upper peripheral pins 334 of the upper peripheral pin subassembly 330 through the upper peripheral pin passage apertures 338 and the lower peripheral pins 344 of the lower peripheral pin subassembly 340 through the lower peripheral pin passage apertures 348. Additionally, a peripheral pin operating inflatable member 320 is arranged to deploy each of the upper central pin subassembly 350 and the lower central pin subassembly 360. A peripheral pin operating inflatable member fill tube 326 provides a filling medium passageway from the intervertebral device body sidewall 308 via a peripheral pin operating inflatable member fill passageway 328 to an interior of the peripheral pin operating inflatable member 320. As the filling medium is transferred to the peripheral pin operating inflatable member 320, the peripheral pin operating inflatable member 320 expands, deploying the lower peripheral pins 344 of the lower peripheral pin subassembly 340 through the lower peripheral pin passage apertures 348 and the lower central pins 364 of the lower central pin subassembly 360 through the lower central pin passage apertures 368. The filling medium can be a gas, a fluid, or any other suitable filling medium.

The upper peripheral pin subassembly 330 can include at least one upper peripheral pin 334. One or more of the at least one upper peripheral pin 334 can include an upper peripheral pin orifice 336 passing therethrough. The lower peripheral pin subassembly 340 can include at least one lower peripheral pin 344. One or more of the at least one lower peripheral pin 344 can include a lower peripheral pin orifice 346 passing therethrough. The upper central pin subassembly 350 can include at least one upper central pin 354. One or more of the at least one upper central pin 354 can include an upper central pin orifice 356 passing therethrough. The lower central pin subassembly 360 can include at least one lower central pin 364. One or more of the at least one lower central pin 364 can include a lower central pin orifice 366 passing therethrough.

The intervertebral device body 302 can include one or more interior chambers to substantially or completely isolate the volume comprising the peripheral pin operating inflatable member 310 from the volume comprising the peripheral pin operating inflatable member 320. The chambers enable dispensing of different materials through the different elements of the intervertebral device 300. For example, one chamber can isolate any fluid communication with the upper peripheral pin subassembly 330 and the lower peripheral pin subassembly 340 and a second chamber can isolate any fluid communication with the upper central pin subassembly 350 and the lower central pin subassembly 360.

The initial insertion process of the intervertebral device 300 would be similar to the initial insertion processes of the intervertebral device 100 and the intervertebral device 200 as described above. The pin subassemblies 330, 340, 350, 360 would be retracted in an insertion configuration. Upon insertion, the central pin subassemblies 350, 360 can be deployed, the peripheral pin subassemblies 330, 340 can be deployed, or all of the pin subassemblies 330, 340, 350, 360 can be deployed. When deployed, the pins 334, 344, 354, 364 would pass through the respective pin passage apertures 338, 348, 358, 368. Deployment of the pin subassemblies 330, 340, 350, 360 can be accomplished by inflating the respective inflatable member 310, 320. In one solution, the respective inflatable member 310, 320 can remain in place. The respective pins 334, 344, 354, 364 of the selected deployed pin subassemblies 330, 340, 350, 360 would be seated into the respective facing surfaces 406, 414 of the adjacent vertebra 402, 412, retaining the intervertebral device 300 in position.

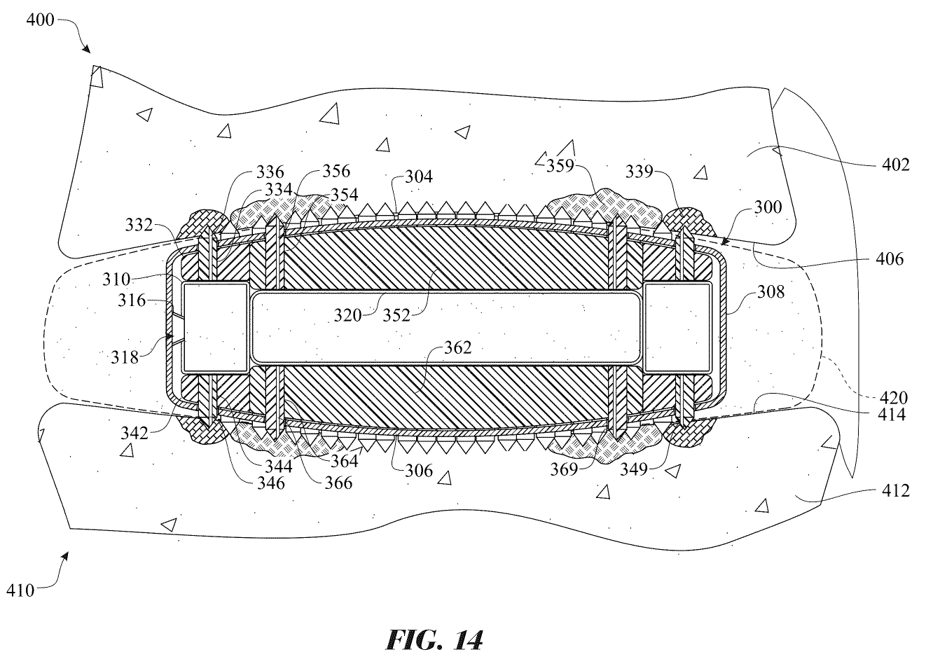

In a second solution, the respective inflatable member 310, 320 can be removed, providing fluid access to optionally include pin orifices 336, 346, 356, 366. Depending upon which pin subassemblies 330, 340, 350, 360 are deployed, the medical professional can dispense a stabilization material through the respective pin orifices 336, 346, 356, 366. In the exemplary illustration presented in FIG. 14, a cement volume 339, 349 can be dispensed through the peripheral pin orifices 336, 346 of the peripheral pin subassemblies 330, 340 and a bone graft deposit 359, 369 can be dispensed through the central pin orifices 356, 366 of the central pin subassemblies 350, 360. The dispensing process can use chambers to isolate the materials.

In an alternative arrangement, the inflatable elements 310, 320 can be replaced with one or more mechanical instruments. For example, a cam located at an end of an elongated rod can be rotated to deploy the peripheral pin subassemblies 330, 340 and/or the central pin subassemblies 350, 360. The instrument would be inserted through a respective port and manipulated to deploy the selected peripheral pin subassemblies 330, 340 and/or the central pin subassemblies 350, 360. The instruments would be removed and the stabilization material would be dispensed into the respective chambers and delivered to into the adjacent vertebra 402, 412 through the respective pin orifices 336, 346, 356, 366 in a manner that is similar to the intervertebral device 100 and the intervertebral device 200 described above.

The exemplary intervertebral device 300 includes a plurality of upper peripheral pin passage apertures 338 extending through an intervertebral device body upper vertebral contacting surface 304 of an intervertebral device body 302 of the intervertebral device 300, a plurality of upper central pin passage apertures 358 extending through the intervertebral device body upper vertebral contacting surface 304 of the intervertebral device body 302 of the intervertebral device 300, a plurality of lower peripheral pin passage apertures 348 extending through an intervertebral device body lower vertebral contacting surface 306 of the intervertebral device body 302 of the intervertebral device 300, and a plurality of lower central pin passage apertures 368 extending through an intervertebral device body lower vertebral contacting surface 306 of the intervertebral device body 302 of the intervertebral device 300.

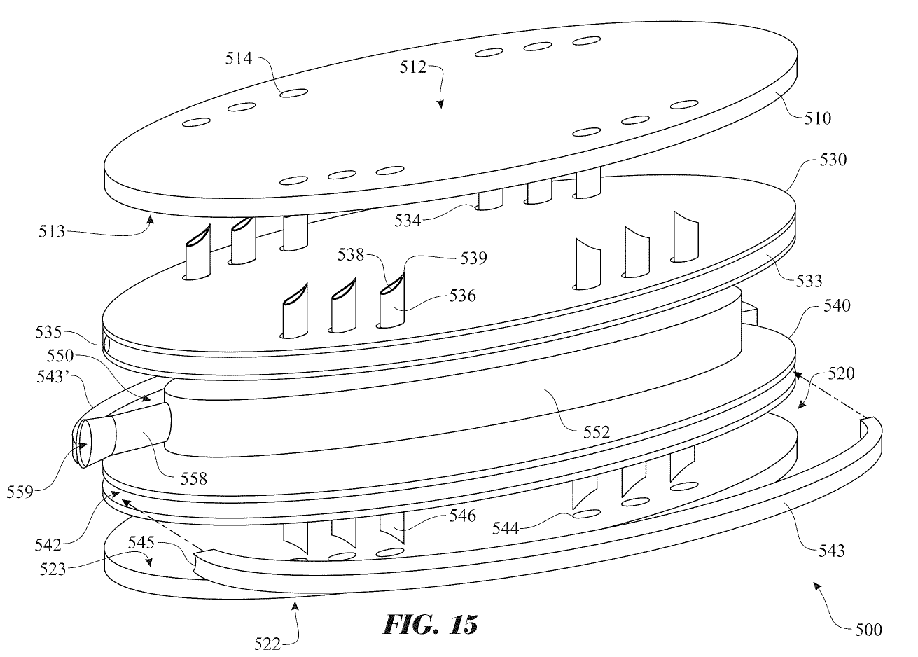

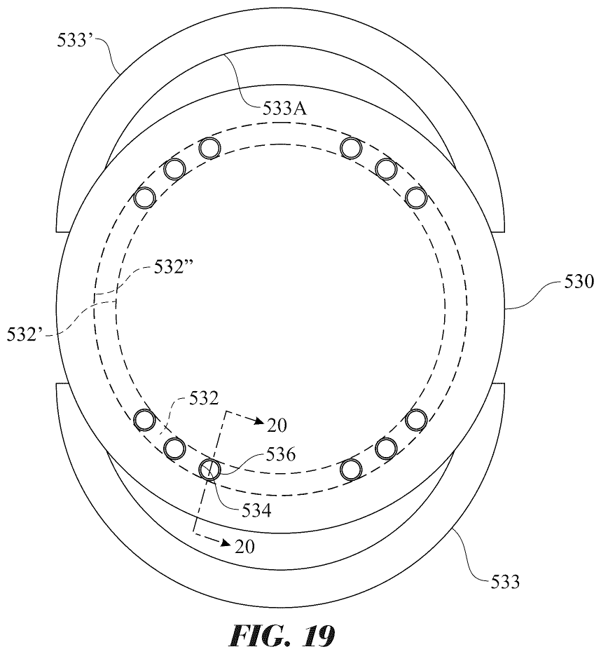

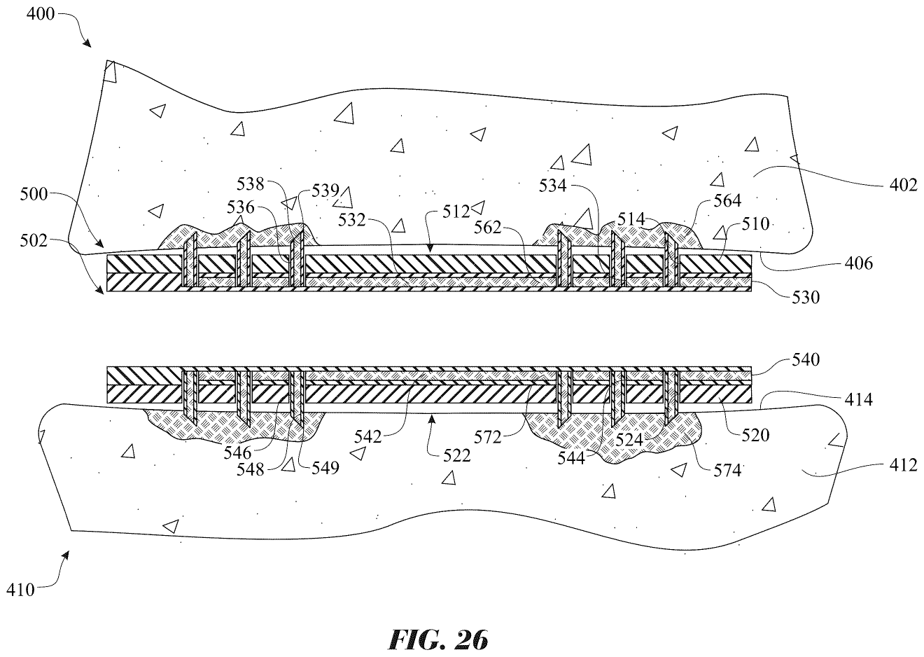

An intervertebral device 500 employs a plurality of upper cannulae 536 assembled to an upper cannula deployment member 530 and, a plurality of lower cannulae 546 assembled to a lower cannula deployment member 540 creating a cannula deployment subassembly 502, the cannula deployment subassembly 502 being enveloped between an intervertebral device upper body 510 and an intervertebral device lower body 520, as illustrated in FIGS. 15 through 18, with FIGS. 19 through 28 illustrating steps of using the exemplary intervertebral device 500. The intervertebral device upper body 510 and the intervertebral device lower body 520 can be in a fixed or moveable relation to one another. In a fixed arrangement, a member can extend in a direction generally perpendicular to the larger plane of each of the intervertebral device upper body 510 and the intervertebral device lower body 520. In one arrangement, the intervertebral device upper body 510 and the intervertebral device lower body 520 can be supported by one or more sidewall members extending between edges of the intervertebral device upper body 510 and the intervertebral device lower body 520. In a second arrangement, the intervertebral device upper body 510 and the intervertebral device lower body 520 can be supported by one or more column members extending between facing surfaces of the intervertebral device upper body 510 and the intervertebral device lower body 520. In a moveable arrangement, any suitable known spacing subsystem can be employed between the intervertebral device upper body 510 and the intervertebral device lower body 520. The spacing subsystem would be designed to accommodate the cannula deployment subassembly 502.

The cannula deployment subassembly 502 comprising a series of upper cannulae 536 carried by an upper cannula deployment member 530 and a series of lower cannulae 546 carried by an intervertebral device lower body 520. A series of upper cannula deployment member cannula supporting bores 534 are formed in the upper cannula deployment member 530 at a desired location to support each respective upper cannula 536.

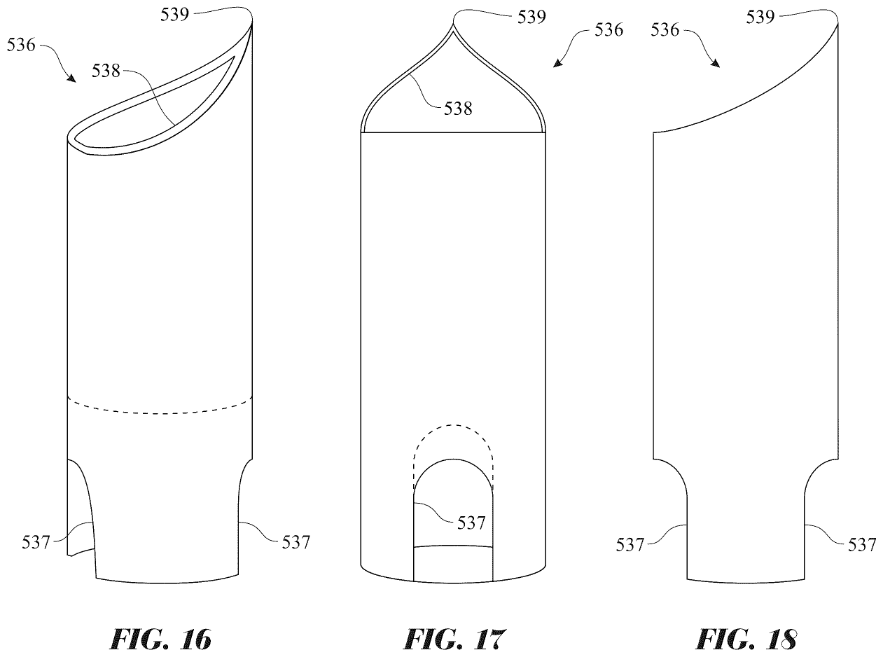

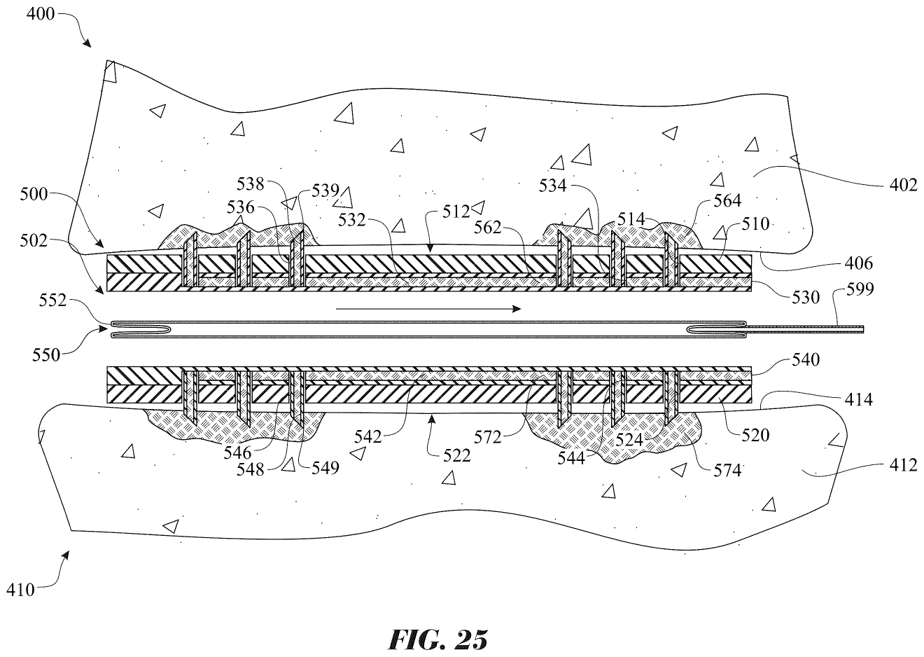

Each upper cannula 536 has a tubular shape (defined by an upper cannula tubular interior surface 538) with one end shaped forming an upper cannula piercing formation 539, as detailed in FIGS. 16 through 18. An upper cannula base fluid passageway 537 is formed at a base end of the upper cannula 536. The upper cannula base fluid passageway 537 is of a size and shape to allow passage of a fluid material from an upper cannula deployment member cement delivery conduit 532 provided within the upper cannula deployment member 530 into the hollowed interior of the upper cannula 536. Each lower cannula 546 is similar to the upper cannula 536, being adapted for integration with the lower cannula deployment member 540. The exemplary illustrations present an upper cannula 536 having a circular cross section shape. The upper cannula 536 can have any suitable shape, with variations to the cross section shape, the impinging tip design, etc. For example, a designer may elect to use an upper cannula 536 having a non-circular cross section shape to ensure the upper cannula 536 remains in a specific rotational orientation throughout the lifespan of the intervertebral device 500. The upper cannula piercing formations 539 may be oriented in different orientations when the upper cannulae 536 are assembled to the upper cannula deployment member 530. The different orientations would be designed to balance out any “pulling” due to the orientation of the different angles of the upper cannula piercing formations 539 of the respective upper cannulae 536. The exemplary upper cannulae 536 can remain implanted into the patient or removed, as illustrated in FIGS. 25 through 28. The materials for each of the elements of the cannula deployment subassembly 502 would be determined by the implementation thereof.

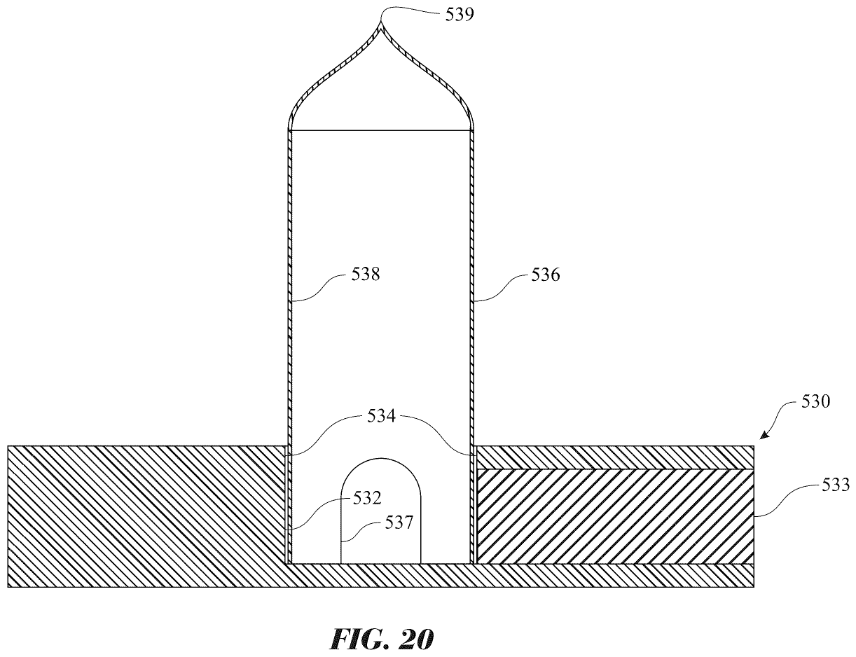

The upper cannula deployment member 530 preferably includes an upper cannula deployment member cement delivery conduit 532 designed to act as a conduit for fluid from a delivery input to each of the upper cannulae 536. In the exemplary illustration, the upper cannula deployment member cement delivery conduit 532 is curved. This complicates options for fabrication of the upper cannula deployment member 530, but increases options for the layout of the upper cannulae 536. In the exemplary illustration, the upper cannula deployment member cement delivery conduit 532 is formed by milling a blind groove terminating at an upper cannula deployment member cement delivery conduit radially inner surface 532′ around a peripheral edge of the upper cannula deployment member 530 to an interior edge of the upper cannula deployment member cannula supporting bore 534, as best illustrated in FIG. 20. An upper cannula deployment member first delivery conduit fill member 533 and an upper cannula deployment member second delivery conduit fill member 533′ are fabricated having an outer peripheral edge that follows a contour of the upper cannula deployment member 530 and an interior peripheral edge delivery conduit fill member interior surface 533A that forms the upper cannula deployment member cement delivery conduit radially inner surface 532′. The upper cannula deployment member first delivery conduit fill member 533 and an upper cannula deployment member second delivery conduit fill member 533′ are installed into the blind groove locating an upper cannula deployment member cement delivery conduit radially outer surface 532″ as illustrated in FIG. 19. When the upper cannula deployment member first delivery conduit fill member 533 and the upper cannula deployment member second delivery conduit fill member 533′ are installed, the delivery conduit fill member interior surface 533A defines an upper cannula deployment member cement delivery conduit radially outer surface 532″ of the upper cannula deployment member cement delivery conduit 532. The upper cannula deployment member cement delivery conduit radially inner surface 532′ and the upper cannula deployment member cement delivery conduit radially outer surface 532″ define the upper cannula deployment member cement delivery conduit 532. A depth and shape of the upper cannula deployment member cement delivery conduit radially inner surface 532′ can vary using computer controlled machining. The upper cannula deployment member first delivery conduit fill member upper cannula deployment member first delivery conduit fill member 533 and the upper cannula deployment member second delivery conduit fill member 533′ can be fabricating using computer controlled machining to shape the delivery conduit fill member interior surface 533A to follow a contour of the upper cannula deployment member cement delivery conduit radially inner surface 532′.

In one alternative fabrication process, the upper cannula deployment member 530 can be fabricated using three dimensional printing techniques with the appropriate materials.

When desired, the upper cannula deployment member cannula supporting bore 534 is formed having a non-circular cross section shape to receive an upper cannula 536 having a compatible, non-circular cross section shape to deter against any rotating motion of the upper cannula 536 within the upper cannula deployment member cannula supporting bore 534. This will maintain the orientation of the upper cannula piercing formation 539 and proper alignment of the upper cannula base fluid passageway 537 to the upper cannula deployment member cement delivery conduit 532.

The upper cannula deployment member cannula supporting bore 534 can be formed in the upper cannula deployment member 530 prior to or following the assembly of the upper cannula deployment member first delivery conduit fill member 533 and the upper cannula deployment member second delivery conduit fill member 533′ to the upper cannula deployment member 530. When formed or following the assembly of the upper cannula deployment member first delivery conduit fill member 533 and the upper cannula deployment member second delivery conduit fill member 533′ to the upper cannula deployment member 530, the bore forming the upper cannula deployment member cannula supporting bore 534 can additionally machine a portion of the upper cannula deployment member delivery conduit fill members 533, 533′.

Each of the plurality of upper cannulae 536 is arranged in vertical registration with a respective intervertebral device body upper vertebral cannula passage 514 of the intervertebral device upper body 510. Similarly, each of the plurality of lower cannulae 546 is arranged in vertical registration with a respective lower cannula deployment member cannula supporting bore 544 of the intervertebral device lower body 520. Each of the plurality of upper cannulae 536 is arranged to be deployed in a first direction and each of the plurality of lower cannulae 546 are arranged to be deployed in a second, opposite direction.

The system is replicated with the lower cannula deployment member 540, wherein the reference numerals of the elements assembled to the intervertebral device upper body 510 are incremented by 10 for elements assembled to the intervertebral device lower body 520.

A cannula deploying subassembly 550 is one exemplary expansion form factor. The exemplary cannula deploying subassembly 550 includes a cannula deploying inflatable member 552 forming a bladder. The cannula deploying subassembly 550 has an exterior surface, defining a cannula deploying inflatable member upper cannula member contacting surface 557 and a cannula deploying inflatable member lower cannula member contacting surface 557′, and an interior surface 554 defining a cannula deploying inflatable member interior volume 555. The cannula deploying inflatable member interior volume 555 is provided to receive at least one of a gas and a fluid, wherein the at least one of the gas and the fluid expands the cannula deploying inflatable member 552, separating the upper cannula deployment member 530 and the lower cannula deployment member 540 from one another. A cannula deploying inflatable member fill element 599 is provided in fluid communication with an interior of the cannula deploying inflatable member 552. The cannula deploying inflatable member fill element 599 is a tubular element enabling passage of at least one of the gas and the fluid therethrough. A valve (not shown but well understood by those skilled in the art) can be provided between the interior of the cannula deploying inflatable member 552 and a tubular passageway of the cannula deploying inflatable member fill element 599. The valve ensures that the at least one of the gas and the fluid dispensed into the cannula deploying inflatable member interior volume 555 remains therein until one desires to remove the at least one of the gas and the fluid therefrom.

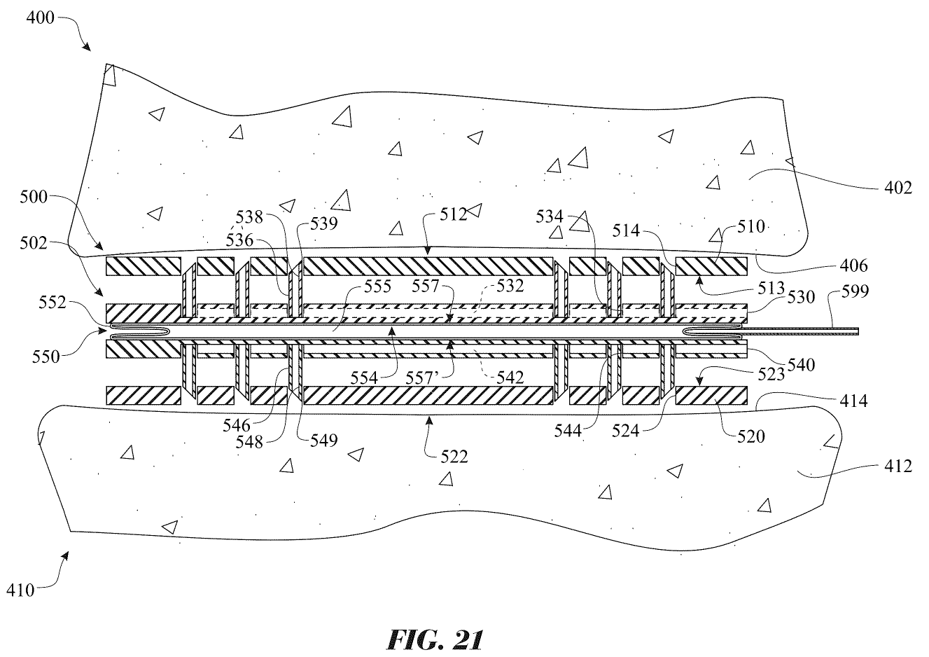

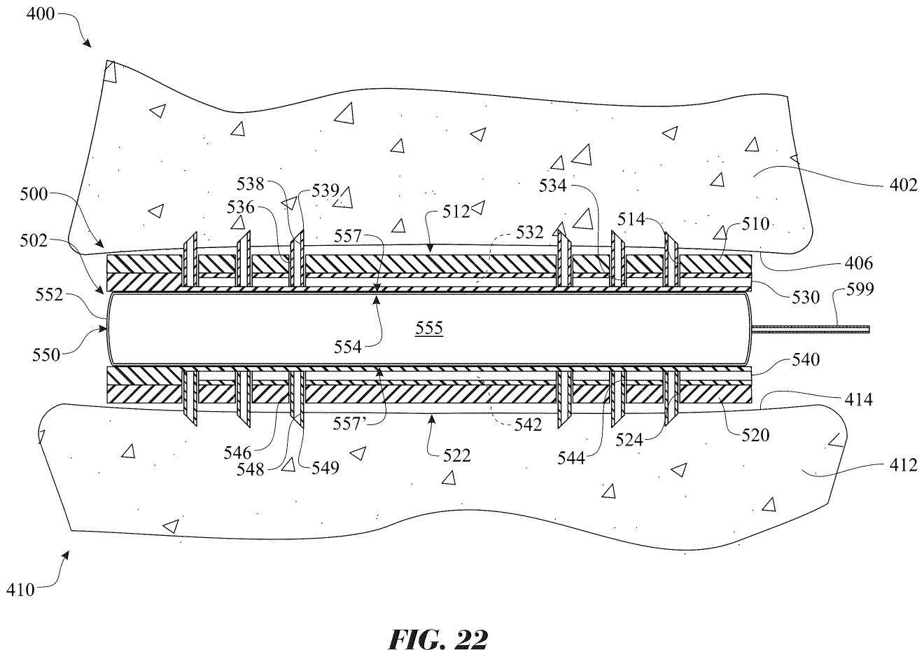

The upper cannula deployment member 530 includes a series of upper cannulae 536 arranged in a pattern preferably following a peripheral contour of the upper cannula deployment member 530. The number and location of the upper cannulae 536 of the series of upper cannulae 536 are such to allow insertion of the cannula deployment subassembly 502 between interior facing surfaces of the intervertebral device upper body 510 and the intervertebral device lower body 520 when retracted (as shown in FIG. 21) and while ensuring that an upper cannula piercing formation 539 and at least a portion of a passageway defined by the upper cannula tubular interior surface 538 encroaches into the first vertebrae 402 when deployed (as shown in FIG. 22). The cannula deployment subassembly 502 can be inserted within the intervertebral device 500 prior to insertion between adjacent vertebras 402, 412 or following insertion of the intervertebral device 500 between adjacent vertebras 402, 412.

Steps of implanting the intervertebral device 500 are illustrated in FIGS. 21 through 24, with optional steps for removal of the cannula deployment subassembly 502 being illustrated in FIGS. 25 through 28.

In the initial step, the cannula deployment subassembly 502 is placed into a retracted configuration (as illustrated in FIG. 21), where the cannula piercing formations 539, 549 are located below an intervertebral device body upper vertebral contacting surface 512 of the intervertebral device upper body 510 and the intervertebral device body lower vertebral contacting surface 522 and the intervertebral device lower body 520, respectively. This allows for insertion of the intervertebral device 500 between the first vertebrae 402 and the second vertebrae 412 without risk of scraping of the surfaces 406, 414 of the respective vertebrae 402, 412 or damage to the sharp formation of each cannula insertion point 529, 549.

Once the intervertebral device 500 is properly positioned, the cannula deploying subassembly 550 can be expanded by filling the cannula deploying inflatable member interior volume 555 with a non-solid material. The cannula deploying subassembly 550 can be filled using any suitable filling arrangement known by those skilled in the art. The exemplary filling arrangement employs a cannula deploying inflatable member fill element 599, which can be inserted through the cannula deploying inflatable member fill feature passageway 559, into the cannula deploying inflatable member fill feature 558. Any suitable valve can be integrated into the cannula deploying inflatable member fill feature 558 to maintain the filling material within the cannula deploying inflatable member interior volume 555. The filling material is transferred through the cannula deploying inflatable member fill element 599 into the cannula deploying inflatable member interior volume 555 of the cannula deploying inflatable member 552, inflating the cannula deploying inflatable member 552 as illustrated in FIG. 22. As the 552 is filled, a cannula deploying inflatable member upper cannula member contacting surface 557 of the cannula deploying inflatable member 552 drives the upper cannula deployment member 530 upward embedding distal portions of the series of upper cannulae 536 through the first vertebrae second joint surface 406 and into the first vertebrae 402 and the cannula deploying inflatable member lower cannula member contacting surface 557′ of the cannula deploying inflatable member 552 drives the lower cannula deployment member 540 downward embedding distal portions of the series of lower cannulae 546 through the second vertebrae first joint surface 414 and into the second vertebrae 412, as illustrated in FIG. 22.

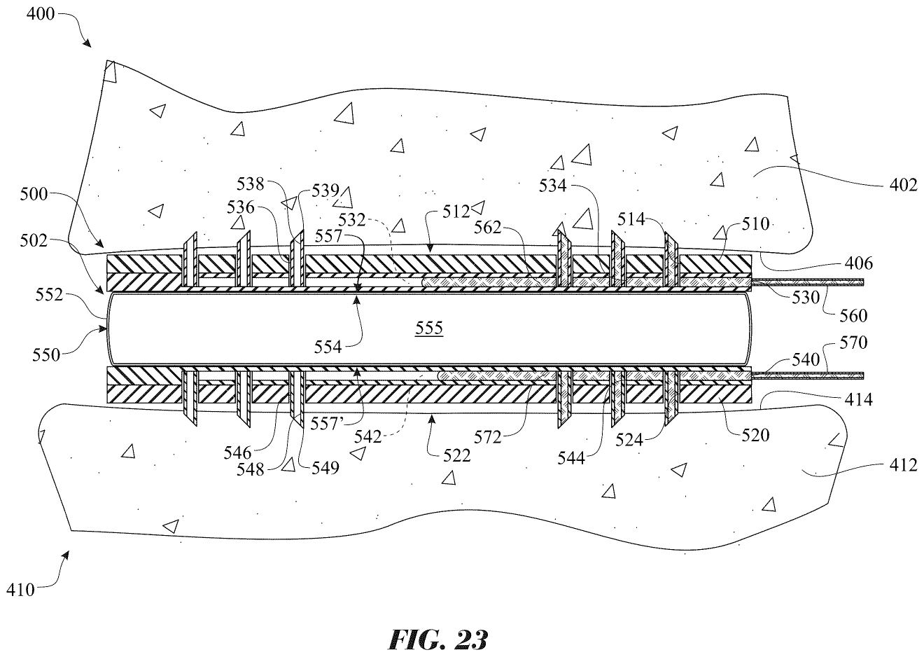

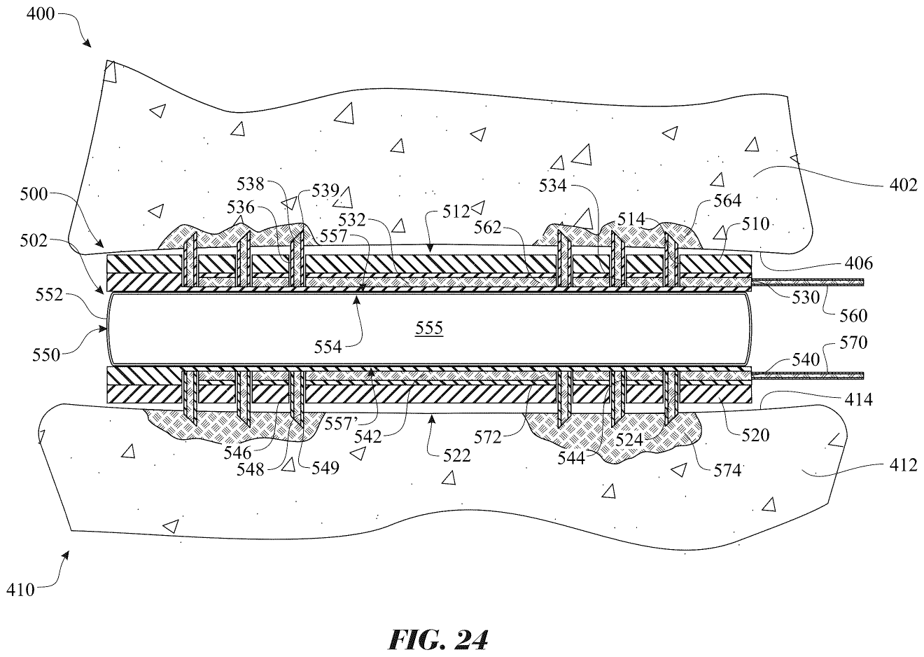

Following embedding of the distal portions of the series of upper cannulae 536 and the distal portions of the series of lower cannulae 546, a cement delivery element (upper delivery portion) 560 is placed into fluid communication with the upper cannula deployment member 530 and, similarly, a cement delivery element (lower delivery portion) 570 is placed into fluid communication with the lower cannula deployment member 540. A bone cement or a bone-cement like compound 562, 572 is conveyed through the cement delivery elements 560, 570, through the upper cannula deployment member cement delivery conduit 532, 542, and continues through each cannula tubular interior 538, 548 until impregnating the respective vertebrae 402, 412, as illustrated in an initial delivery condition in FIG. 23 and a completed delivery condition in FIG. 24. Delivery of the bone cement or a bone-cement like compound leaves a dispensed cement (within bone) 564 within the first vertebrae 402 and a dispensed cement (within bone) 574 within the second vertebrae 412. The bone cement or bone-cement like compound provides structural support while either possessing properties that either stimulate bone growth, allow resorption, or both.

Following the process of impregnating the respective vertebrae 402, 412 with the bone cement or a bone-cement like compound, the cannula deployment subassembly 502 can either remain in place or be removed.

In an option where the cannula deployment subassembly 502 remains in situ, the cannula deploying inflatable member fill element 599 can be removed and the valve would retain the expansion fluid within the cannula deploying inflatable member interior volume 555. Alternatively, the cannula deploying inflatable member fill element 599 can be used to withdraw the inflating material from the cannula deploying inflatable member interior volume 555 of the cannula deploying inflatable member 552 and the contracted cannula deploying inflatable member 552 can be removed, as illustrated in FIGS. 24 and 25. The upper cannula deployment member 530 and the lower cannula deployment member 540 would remain in position by the bone cement or a bone-cement like compound.

Optional steps for removing the cannula deployment subassembly 502 are illustrated in FIGS. 25 through 28. A first removal step contracts the expanded cannula deploying inflatable member 552, as illustrated in FIG. 25. This can be accomplished by withdrawing the inflating material from the cannula deploying inflatable member interior volume 555 of the cannula deploying inflatable member 552 using the cannula deploying inflatable member fill element 599, as described above. The removed cannula deploying inflatable member 552 provides a clearance for removal of the upper cannula deployment member 530 and the lower cannula deployment member 540. Removal of the upper cannula deployment member 530 is described and illustrated herein and representative of the steps for removal of the lower cannula deployment member 540. In a condition where the plurality of cannulae 536, 546 are removed, the residual bone cement can create cement retention formations (within intervertebral device) 566.

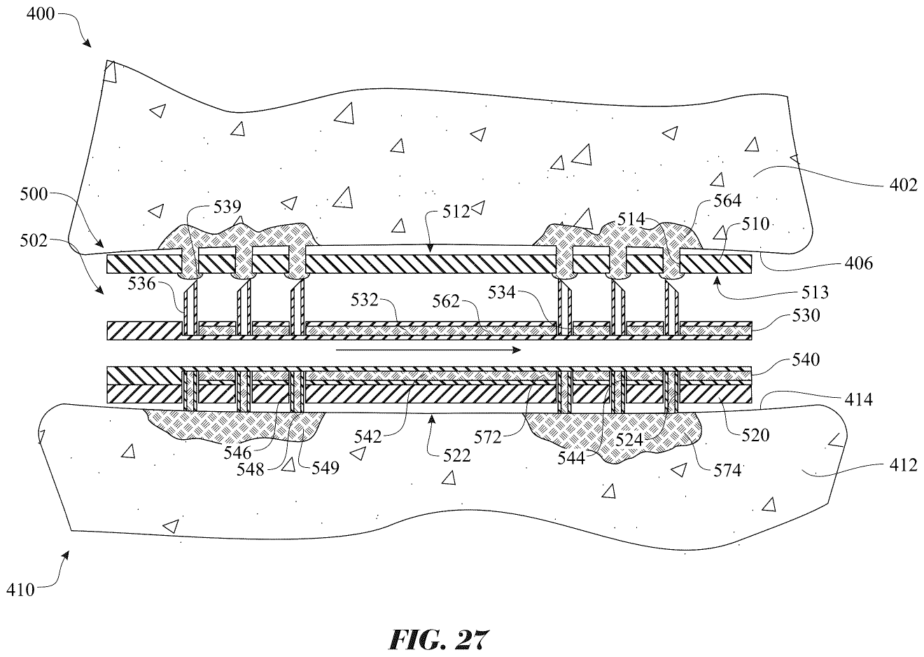

The medical professional would draw the upper cannula deployment member 530 downward until the upper cannula piercing formations 539 are all clear of an intervertebral device body upper vertebral interior surface 513 of the intervertebral device upper body 510, as illustrated in FIG. 27. This can be accomplished using any suitable instrument(s). After the upper cannula piercing formations 539 all clear the intervertebral device body upper vertebral interior surface 513, the upper cannula deployment member 530 can be removed from the intervertebral device 500. In an alternate process, the series of upper cannula 536 can remain within each respective intervertebral device body upper vertebral cannula passage 514. This can improve a mechanical assembly between the intervertebral device upper body 510 and the first vertebrae 402. The dimensions of the interior of the upper cannula deployment member cannula supporting bore 534 respective to dimensions of the exterior of the upper cannula 536 can be designed to either retain or release each upper cannula 536 from each upper cannula deployment member cannula supporting bore 534.

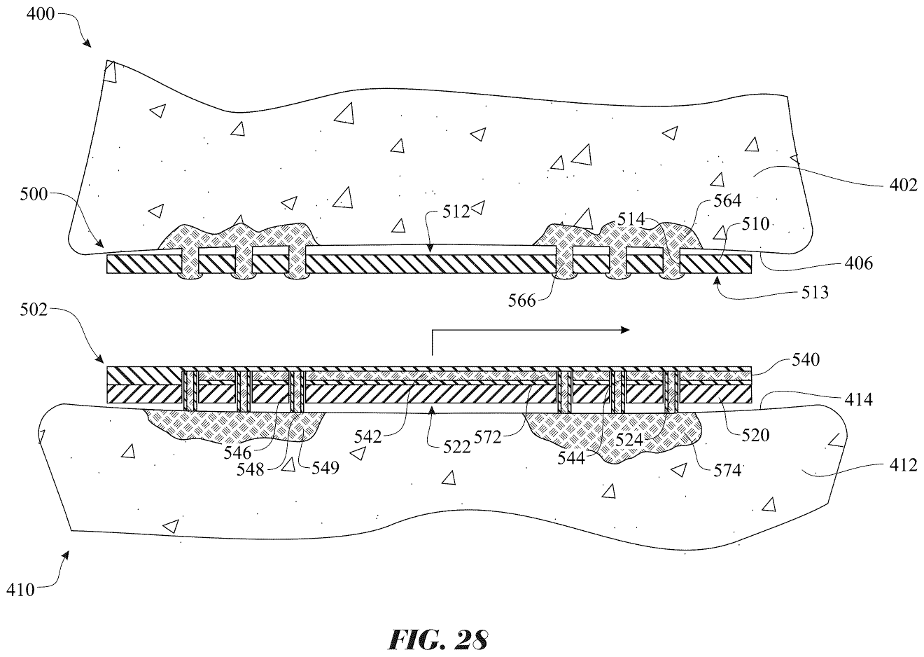

The process would be repeated to remove the lower cannula deployment member 540 from the intervertebral device 500, as illustrated in FIG. 28.

The intervertebral device 500 has generally planar upper and lower intervertebral device bodies 510, 520; each having a generally planar contacting surface 512, 522 and a generally planar facing surface 513, 523. Similarly, each upper cannula deployment member 530 and lower cannula deployment member 540 is generally planar in shape. In certain applications, a variant of the intervertebral device 500 may be more appropriate for implanting.

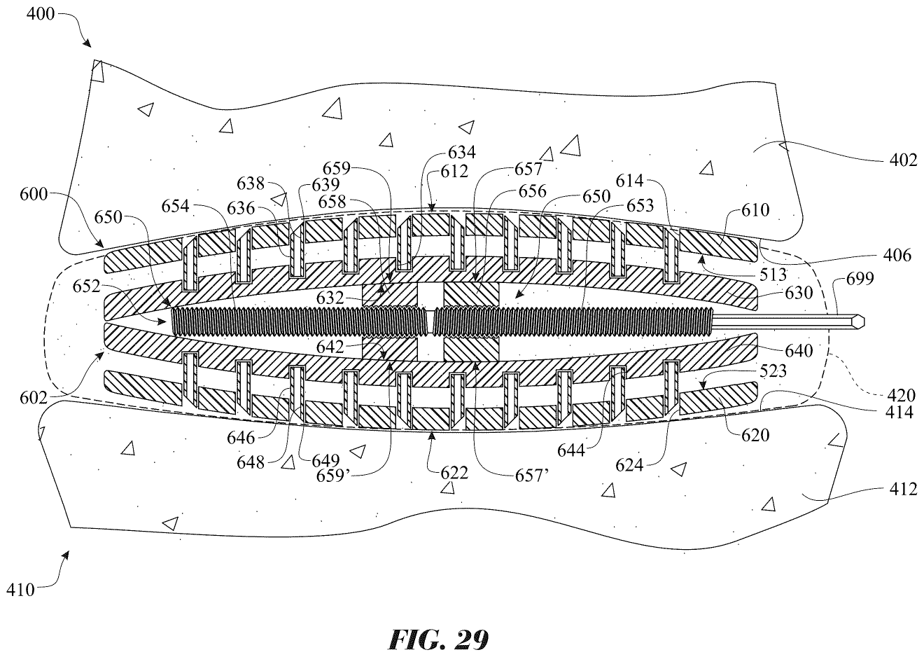

An intervertebral device 600 is similar to the intervertebral device 500 with variations as described herein; the intervertebral device 600 being illustrated in FIGS. 28 and 29. The intervertebral device 600 and the intervertebral device 500 have a majority of like elements. Like elements of the intervertebral device 500 and the intervertebral device 600 are numbered the same, wherein elements of the intervertebral device 600 are preceded by the numeral “6”. The intervertebral device 600 includes an intervertebral device upper body 610 and an intervertebral device lower body 620, each having an arched contacting surface 612, 622 and an arched facing surface 613, 623.

A cannula deployment subassembly 602 is located between the intervertebral device upper body 610 and the intervertebral device lower body 620. The cannula deployment subassembly 602 includes elements of the cannula deployment subassembly 502 as described above. In the intervertebral device 600, the cannula deploying subassembly 550 is replaced with a cannula deploying subassembly 650. The exemplary cannula deploying subassembly 550 employs an inflating fluid. Conversely, the exemplary cannula deploying subassembly 650 is a mechanical expanding system. The cannula deploying subassembly 650 includes a cannula deploying threaded control rod 652 having a cannula deploying threaded control rod first rotational direction threaded section 653 located along a first portion thereof and a cannula deploying threaded control rod second, opposite rotational direction threaded section 654 located along a second portion thereof. A cannula deploying first wedge member 656 is threadably assembled to the cannula deploying threaded control rod first rotational direction threaded section 653 of the cannula deploying subassembly 650. An outer surface of the cannula deploying first wedge member 656 slideably engages with an upper cannula deployment member wedge contacting surface 632 of the upper cannula deployment member 630 and a lower cannula deployment member wedge contacting surface 642 of the lower cannula deployment member 640. Similarly, a cannula deploying second wedge member 658 is threadably assembled to the cannula deploying threaded control rod second, opposite rotational direction threaded section 654 of the cannula deploying subassembly 650.

The arched shape allows for a reduced height of the cannulae 636, 646, as the shape of the intervertebral device body members 610, 620 follow a contour of the contacting surfaces 406, 414 of the respective vertebra 402, 412.

During installation, the cannula deployment subassembly 602 is positioned in a retracted configuration.

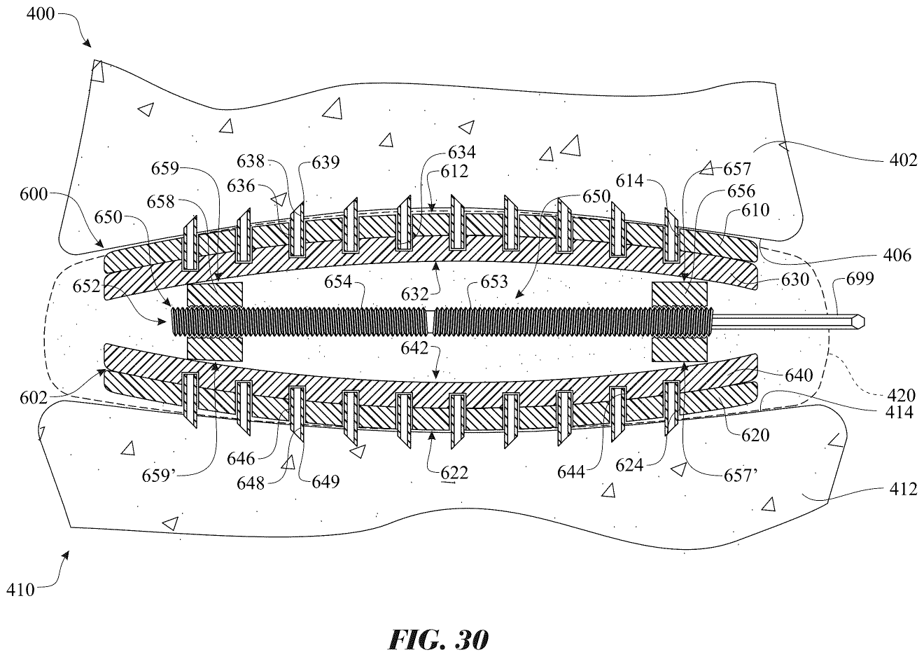

During use, a cannula deploying threaded control rod operating tool 699 engages with the cannula deploying threaded control rod 652. The cannula deploying threaded control rod operating tool 699 is rotated (either by an operator or a motor), driving a rotation of the cannula deploying threaded control rod 652. The rotating motion of the cannula deploying threaded control rod 652 rotates each of the cannula deploying threaded control rod first rotational direction threaded section 653 and the cannula deploying threaded control rod second, opposite rotational direction threaded section 654. The opposing thread design separates the cannula deploying first wedge member 656 and cannula deploying second wedge member 658 from one another. A combination of the arched shape of the intervertebral device body upper vertebral interior surface 613 and the intervertebral device body lower vertebral interior surface 623 and the movements of the cannula deploying first wedge member 656 and the cannula deploying second wedge member 658 drive the upper cannula deployment member 630 and the lower cannula deployment member 640 apart from one another, as illustrated in FIG. 30. A cannula deploying first wedge upper cannula member contacting surface 657 of the cannula deploying first wedge member 656 slideably engages with the upper cannula deployment member wedge contacting surface 632 of the upper cannula deployment member 630 and a cannula deploying first wedge lower cannula member contacting surface 657′ of the cannula deploying first wedge member 656 slideably engages with the lower cannula deployment member wedge contacting surface 642 of the lower cannula deployment member 640.

Similarly, a cannula deploying second wedge upper cannula member contacting surface 659 of the cannula deploying second wedge member 658 slideably engages with the upper cannula deployment member wedge contacting surface 632 of the upper cannula deployment member 630 and a cannula deploying second wedge lower cannula member contacting surface 659′ of the cannula deploying second wedge member 658 slideably engages with the lower cannula deployment member wedge contacting surface 642 of the lower cannula deployment member 640.

Separation between the upper cannula deployment member 630 and the lower cannula deployment member 640 drive a series of upper cannulae 636 upward through an intervertebral device body upper vertebral cannula passage 614 of the intervertebral device upper body 610 and a series of lower cannulae 646 downward through an intervertebral device body lower vertebral cannula passage 624 of the intervertebral device lower body 620. Additional details of the process replicate the same deployment process of the intervertebral device 500 as described above.

Portions of features of the intervertebral device 500 and the intervertebral device 600 can be integrated within the other of the intervertebral device 500 and the intervertebral device 600. In a first example, the intervertebral device upper body 510 and the intervertebral device lower body 520 can be arched in shape. In a second example, the cannula deploying subassembly 650 can be replaced with an inflatable cannula deploying subassembly 550. In a third example, the intervertebral device body vertebral contacting surfaces 512, 522 can be planar in shape and the intervertebral device body vertebral interior surfaces 513, 523 can be arched or wedged shaped, employing the cannula deploying subassembly 650,

The above-described embodiments are merely exemplary illustrations of implementations set forth for a clear understanding of the principles of the invention. Many variations, combinations, modifications or equivalents may be substituted for elements thereof without departing from the scope of the invention. Therefore, it is intended that the invention not be limited to the particular embodiments disclosed as the best mode contemplated for carrying out this invention, but that the invention will include all the embodiments falling within the scope of the appended claims.

### ELEMENT DESCRIPTIONS

### Ref. No. Description

100 intervertebral device
102 intervertebral device body
104 intervertebral device body upper vertebral contacting surface
106 intervertebral device body lower vertebral contacting surface
108 intervertebral device body sidewall
110 interbody lock actuating member
112 interbody lock actuating member translating end
114 interbody lock actuating member actuating end
116 interbody lock actuating control conduit
118 interbody lock actuating control conduit opening
120 deployable interbody locking member
121 deployable interbody locking member passageway
122 deployable interbody locking member translating end
124 deployable interbody locking member actuating end
126 deployable interbody locking member guide
128 deployable interbody locking member guide opening
139 stabilization material
200 intervertebral device
202 intervertebral device body
204 intervertebral device body upper vertebral contacting surface
206 intervertebral device body lower vertebral contacting surface
208 intervertebral device body sidewall
220 interbody locking cannula
220′ interbody locking cannula (inserted)
222 interbody locking cannula actuating end
224 interbody locking cannula engaging end
224′ interbody locking cannula engaging end (inserted)
226 interbody lock actuating control conduit
228 interbody lock actuating control conduit actuating opening
229 interbody lock actuating control conduit engaging opening
239 stabilization material
300 intervertebral device
302 intervertebral device body
304 intervertebral device body upper vertebral contacting surface
306 intervertebral device body lower vertebral contacting surface
308 intervertebral device body sidewall
310 peripheral pin operating inflatable member
316 peripheral pin operating inflatable member fill tube
318 peripheral pin operating inflatable member fill passageway
320 peripheral pin operating inflatable member
326 peripheral pin operating inflatable member fill tube
328 peripheral pin operating inflatable member fill passageway
330 upper peripheral pin subassembly
332 upper peripheral pin supporting base
334 upper peripheral pins
336 upper peripheral pin orifice
338 upper peripheral pin passage apertures
339 upper cement volume
340 lower peripheral pin subassembly
342 lower peripheral pin supporting base
344 lower peripheral pins
346 lower peripheral pin orifice
348 lower peripheral pin passage apertures
349 lower cement volume
350 upper central pin subassembly
352 upper central pin support base
354 upper central pins
356 upper central pin orifice
358 upper central pin passage apertures
359 upper bone graft deposit
360 lower central pin subassembly
362 lower central pin support base
364 lower central pins
366 lower central pin orifice
368 lower central pin passage apertures
369 lower bone graft deposit
400 first joint member
402 first vertebrae
404 first vertebrae first joint surface
406 first vertebrae second joint surface
410 second joint member
412 second vertebrae
414 second vertebrae first joint surface
416 second vertebrae second joint surface
420 intra-vertebral disc
450 longitudinal axis
452 lateral axis
500 intervertebral device
502 cannula deployment subassembly
510 intervertebral device upper body
512 intervertebral device body upper vertebral contacting surface
513 intervertebral device body upper vertebral interior surface
514 intervertebral device body upper vertebral cannula passage
520 intervertebral device lower body
522 intervertebral device body lower vertebral contacting surface
523 intervertebral device body lower vertebral interior surface
524 intervertebral device body lower vertebral cannula passage
530 upper cannula deployment member
532 upper cannula deployment member cement delivery conduit
532′ upper cannula deployment member cement delivery conduit radially inner surface
532″ upper cannula deployment member cement delivery conduit radially outer surface
533 upper cannula deployment member first delivery conduit fill member
533′ upper cannula deployment member second delivery conduit fill member
533A delivery conduit fill member interior surface
534 upper cannula deployment member cannula supporting bore
536 upper cannula
538 upper cannula tubular interior surface
537 upper cannula base fluid passageway
539 upper cannula piercing formation
540 lower cannula deployment member
542 lower cannula deployment member cement delivery conduit
544 lower cannula deployment member cannula supporting bore
546 lower cannula
548 lower cannula tubular interior surface
549 lower cannula piercing formation
550 cannula deploying subassembly
552 cannula deploying inflatable member
554 cannula deploying inflatable member interior surface
555 cannula deploying inflatable member interior volume
557 cannula deploying inflatable member upper cannula member contacting surface
557′ cannula deploying inflatable member lower cannula member contacting surface
558 cannula deploying inflatable member fill feature
559 cannula deploying inflatable member fill feature passageway
560 cement delivery element (upper delivery portion)
562 cement delivery (upper delivery portion)
564 dispensed cement (within bone)
566 cement retention formation (within intervertebral device)
570 cement delivery element (lower delivery portion)
572 cement delivery (lower delivery portion)
574 dispensed cement (within bone)
599 cannula deploying inflatable member fill element
600 intervertebral device
602 cannula deployment subassembly
610 intervertebral device upper body
612 intervertebral device body upper vertebral contacting surface
613 intervertebral device body upper vertebral interior surface
614 intervertebral device body upper vertebral cannula passage
620 intervertebral device lower body
622 intervertebral device body lower vertebral contacting surface
623 intervertebral device body lower vertebral interior surface
624 intervertebral device body lower vertebral cannula passage
630 upper cannula deployment member
632 upper cannula deployment member wedge contacting surface
634 upper cannula deployment member cannula supporting bore
636 upper cannula
638 upper cannula interior surface
639 upper cannula insertion point
640 lower cannula deployment member
642 lower cannula deployment member wedge contacting surface
644 lower cannula deployment member cannula supporting bore
646 lower cannula
648 lower cannula interior surface
649 lower cannula insertion point
650 cannula deploying subassembly
652 cannula deploying threaded control rod
653 cannula deploying threaded control rod first rotational direction threaded section
654 cannula deploying threaded control rod second, opposite rotational direction threaded section
656 cannula deploying first wedge member
657 cannula deploying first wedge upper cannula member contacting surface
657′ cannula deploying first wedge lower cannula member contacting surface
658 cannula deploying second wedge member
659 cannula deploying second wedge upper cannula member contacting surface
659′ cannula deploying second wedge lower cannula member contacting surface
699 cannula deploying threaded control rod operating tool

What is claimed is:

1. A method for fusing adjacent vertebra, the method comprising steps of:
inserting an implantable vertebral interbody device between adjacent vertebras, the implantable vertebral interbody device comprising:
a body having an upper vertebral contacting surface, a lower vertebral contacting surface, and a sidewall extending between the upper vertebral contacting surface and the lower vertebral contacting surface,
a port through the sidewall,
at least one orifice passing through at least one of the upper vertebral contacting surface and the lower vertebral contacting surface; and
a moveable cannula passing through a respective orifice of the at least one orifice passing through the at least one of the upper vertebral contacting surface and the lower vertebral contacting surface;
deploying the cannula by applying a force to a first, force receiving end of the cannula, wherein the force inserts a second, engaging end of the cannula into a facing surface of the respective vertebra; and
dispensing a stabilization material through the cannula.

2. The method for fusing adjacent vertebra as recited in claim 1, wherein the step of dispensing a stabilization material through the cannula dispenses the stabilization material into the respective vertebra.

3. The method for fusing adjacent vertebra as recited in claim 1, wherein the step of dispensing a stabilization material through the cannula dispenses the stabilization material into the respective vertebra, wherein the stabilization material stabilizes the respective vertebra in an area proximate the respective surface of the implantable vertebral interbody device.

4. The method for fusing adjacent vertebra as recited in claim 1, wherein the step of dispensing the stabilization material through the cannula is accomplished by using bone cement as the stabilization material.

5. The method for fusing adjacent vertebra as recited in claim 1, the method further comprising a step of:
withdrawing the cannula from the vertebra.

6. The method for fusing adjacent vertebra as recited in claim 1, the method further comprising a step of:
passing the cannula through the port through the sidewall.

7. The method for fusing adjacent vertebra as recited in claim 1, wherein the step of deploying the cannula by applying a force to a first, force receiving end of the cannula is accomplished by utilizing a mechanically applied force.

8. The method for fusing adjacent vertebra as recited in claim 1, wherein the step of deploying the cannula by applying a force to a first, force receiving end of the cannula is accomplished by utilizing a pneumatically applied force.

9. The method for fusing adjacent vertebra as recited in claim 1, the second, engaging end of the cannula further comprising a pointed shape, the method further comprising a step of:
using the pointed shape of the cannula to penetrate the respective vertebra of the adjacent vertebras.

10. The method for fusing adjacent vertebra as recited in claim 1, the implantable vertebral interbody device comprising further comprising a cannula deploying member, the cannula deploying member being engaged with the cannula, the method further comprising a step of:
using the cannula deploying member to apply the force to a first, force receiving end of the cannula.

11. The method for fusing adjacent vertebra as recited in claim 1, the implantable vertebral interbody device comprising further comprising a cannula deploying member, the cannula deploying member being engaged with the cannula, the method further comprising a step of:
using the cannula deploying member to apply the force to a first, force receiving end of the cannula.

12. The method for fusing adjacent vertebra as recited in claim 1, the implantable vertebral interbody device further comprising at least one upper cannula arranged to pass through one of the upper vertebral contacting surface and the lower vertebral contacting surface, the method further comprising steps of:
applying an outwardly directed vertical force to each force receiving end of each at least one upper cannula to penetrate the respective vertebra of the adjacent vertebras; and
applying a downward force to each force receiving end of each at least one lower cannula to penetrate the respective vertebra of the adjacent vertebras.

13. The method for fusing adjacent vertebra as recited in claim 1, the implantable vertebral interbody device further comprising at least one upper cannula arranged to pass through the upper vertebral contacting surface and at least one lower cannula arranged to pass through the lower vertebral contacting surface, the method further comprising steps of:
applying an upward force to each force receiving end of each at least one upper cannula to penetrate the respective vertebra of the adjacent vertebras; and
applying a downward force to each force receiving end of each at least one lower cannula to penetrate the respective vertebra of the adjacent vertebras.

14. The method for fusing adjacent vertebra as recited in claim 1, the implantable vertebral interbody device further comprising a cannula subassembly comprising at least one cannula carried by a cannula supporting base, each at least one cannula being in registration with a respective aperture of the at least one aperture, the method further comprising a step of:
outwardly positioning the pin subassembly by one of a mechanical device inserted through the port or a pressure generating device operated through the port.

15. The implantable vertebral interbody device used in the method as recited in claim 1.

16. The implantable vertebral interbody device used in the method as recited in claim 1, wherein the cannula additionally passes through the port through the sidewall.

17. The implantable vertebral interbody device used in the method as recited in claim 1, the second, engaging end of the cannula further comprising a pointed shape.

18. The implantable vertebral interbody device used in the method as recited in claim 1, the upper vertebral contacting surface and the lower vertebral contacting surface having a planar horizontal surface.

19. The implantable vertebral interbody device used in the method as recited in claim 1, the upper vertebral contacting surface and the lower vertebral contacting surface having an outwardly convex surface.

20. The implantable vertebral interbody device used in the method as recited in claim 1, at least one element of the implantable vertebral interbody device further comprising a conduit providing transfer of the stabilization material to the cannula.
